(19) United States
(12) Patent Application Publication
(10) Pub. No.: US 2024/0117442 A1
(43) Pub. Date: Apr. 11, 2024

(54) Apparatus and Methods for Analysis of Gene Mutation

(71) Applicant: Postech Research and Business Development Foundation (POSTECH), Pohang (KR)

(72) Inventors: Joon Won Park, Pohang (KR); Sourav Mishra, Pohang (KR); Changill Ban, Pohang (KR)

(73) Assignee: Postech Research and Business Development Foundation (POSTECH), Pohang (KR)

(21) Appl. No.: 18/490,116

(22) Filed: Oct. 19, 2023

Related U.S. Application Data
(63) Continuation-in-part of application No. PCT/IB2021/053355, filed on Apr. 23, 2021.

(51) Int. Cl. C12Q 1/6886

(52) U.S. Cl. CPC C12Q 1/6886; C12Q 2600/156; C12Q 2600/166

(57) ABSTRACT

Methods and apparatuses are disclosed for detecting a presence of a mismatched pair in an oligonucleotide duplex that is attached to a solid substrate using an atomic force microscope. In particular, methods and apparatuses of the invention allow qualitative and quantitative analysis of the presence of a mismatched pair in a sample of oligonucleotide duplex using an atomic force microscope comprising an AFM cantilever that includes a DNA mismatch repair protein. Methods and apparatuses of the invention allow detection of gene mutation without a need for amplification, labeling, or modification of the sample. Such apparatuses and methods can be used in a wide variety of clinical diagnostic applications including detection and/or analysis of biomarkers related to, but not limited to, cancer, trauma, sepsis, aseptic inflammation, myocardial infarction, stroke, transplantation, diabetes, sickle cell disease, as well as other clinical conditions.

### CROSS-REFERENCE TO RELATED APPLICATIONS

This application is a continuation-in-part application of PCT Patent Application No. PCT/IB2021/053355, filed Apr. 23, 2021, which is incorporated by reference herein in its entirety.

### FIELD OF THE INVENTION

The present invention relates to apparatuses and methods for detecting a presence of a mismatched pair in an oligonucleotide duplex that is attached to a solid substrate using an atomic force microscope. In particular, methods and apparatuses of the invention allow qualitative and quantitative analysis of the presence of a mismatched pair in a sample of oligonucleotide duplex by using an atomic force microscope comprising an AFM cantilever that includes a DNA mismatch repair protein. The methods and apparatuses of the invention allow detection of gene mutation without a need for amplification, labeling, or modification of the sample. Such apparatuses and methods can be used in a wide variety of clinical diagnostic applications including detection and/or analysis of biomarkers related to, but not limited to, cancer, trauma, sepsis, aseptic inflammation, myocardial infarction, stroke, transplantation, diabetes, sickle cell disease, as well as other clinical conditions.

### BACKGROUND OF THE INVENTION

Circulating free DNA or cell-free DNA (cfDNA) are degraded DNA fragments released to the blood plasma. Exemplary cfDNAs include, but are not limited to, circulating tumor DNA (ctDNA) and cell-free fetal DNA (cffDNA). Of particular note, elevated levels of cfDNA have been observed in cancer, especially in advanced stage of the disease. There is also evidence that cfDNA increases with the onset of age.

cfDNA has been shown to be a useful biomarker for a variety of clinical conditions including, but not limited to, cancer, trauma, sepsis, aseptic inflammation, myocardial infarction, stroke, transplantation, diabetes, sickle cell disease, as well as other clinical conditions. Other useful cfDNAs include cffDNA for determining not only whether a woman is pregnant but also to determine the presence of any fetus anomaly. cfDNA is primarily a double-stranded extracellular molecule of DNA, consisting of small fragments (70 to 200 bp) and larger fragments (21 kb).

As expected, cell-free DNA analysis, such as cell-free circulating tumor DNA (ctDNA) analysis, provides enormous opportunities for noninvasive early assessment of cancers such as, but not limited to, prostate cancer, breast cancer, colon cancer, as well as other solid tumors. Recent technological advances in ctDNA analysis have shown that liquid biopsy tools, with enhanced limit of detection (LOD) and sensitivity/specificity, can greatly contribute to the diagnostic and prognostic aspects of oncology.

Currently, PCR-based methods have led this field and the limit of detections (“LODs”) achieved are encouraging. Unfortunately, however, PCR-based methods introduce its own mutation during amplification steps and undesirable artifacts during the data analysis resulting in less than ideal sensitivity and/or specificity.

Therefore, there is a need to improve the sensitivity and/or specificity of determining a presence of any anomaly in cfDNA without using PCR-based methods. In particular, there is a need for an apparatus and a method for detecting a gene mutation without a need for amplification, labeling, or modification.

### SUMMARY OF THE INVENTION

Some aspects of the invention are based on the discovery by the present inventors that an atomic force microscope (AFM) in which its cantilever tip comprises a DNA mismatch repair protein provides an extremely sensitive and selective detection of a mismatched oligonucleotide duplex without any labeling, amplification (e.g., via PCR), or modification. Methods and apparatuses of the invention allow both quantitative and qualitative analysis for determining the presence of a mismatched oligonucleotide duplex.

One particular aspect of the invention provides a method for determining a presence of mismatch pair in an oligonucleotide duplex that is attached to a solid substrate, said method comprising:
scanning said solid substrate with an atomic force microscope (AFM) having an AFM tip comprising a DNA mismatch repair protein to produce a force map; and
analyzing said force map to determine the presence of mismatch in said oligonucleotide duplex.

In some embodiments, the method is used to determine the level of mismatched oligonucleotide duplex in a sample. Yet in other embodiments, said DNA mismatch repair protein is a prokaryotic mismatch repair protein. In some instances, said DNA mismatch repair protein comprises MutS or a homolog thereof.

Still in other embodiments, said DNA mismatch repair protein is a eukaryotic mismatch repair protein. In some instances, said DNA mismatch repair protein comprises MSH2, MSH3, MSH4, or MSH6.

Another aspect of the invention provides an atomic force microscope (AFM) cantilever tip comprising a histidine-tagged DNA mismatch repair protein. In some embodiments, said DNA mismatch repair protein is a prokaryotic mismatch repair protein. In one particular instance, said DNA mismatch repair protein comprises MutS or a homolog thereof.

Yet in other embodiments, said DNA mismatch repair protein is a eukaryotic mismatch repair protein. In one particular embodiment, said DNA mismatch repair protein comprises MSH2, MSH3, MSH4, or MSH6.

Still in other embodiments, said histidine-tagged DNA mismatch repair protein is attached to said AFM cantilever tip via a linker.

In certain embodiments, said histidine-tagged DNA mismatch repair protein is immobilized to said AFM cantilever tip by complexation of said histidine-tag with a Ni(II) ion.

Yet another aspect of the invention provides a method for detecting a presence of gene mutation in a sample, said method comprising:
contacting said sample with a solid substrate comprising a probe oligonucleotide under conditions sufficient to form a target-probe oligonucleotide duplex, wherein said probe oligonucleotide comprises a complementary oligonucleotide sequence of a wild-type gene;
measuring a level of interaction between said target-probe oligonucleotide duplex and a DNA mismatch repair protein using an atomic force microscope (AFM); and
analyzing said level of interaction to determine the presence of a mismatched target-probe oligonucleotide duplex,
   wherein the presence of mismatched target-probe oligonucleotide duplex is indication of a presence of gene mutation in said sample. The term “wild-type gene” refers to a normal phenotype that is present in the majority of the natural population, i.e., characteristic that is observed in more than 50% of the population. Alternatively, the term refers to a gene that does not manifest itself as a disease, disorder, or any other clinical condition that is considered abnormal.

In some embodiments, said probe oligonucleotide comprises a complementary oligonucleotide of a wild-type gene selected from the group consisting of Ras, EGFR, and PIK3CA. In some instances, said Ras gene is selected from the group consisting of KRas, HRas, NRas, R-Ras, M-Ras, E-ras, Di-Ras1, Di-Ras2, NKIRas1, NKIRas2, TC21, Rap1, Rap2, Rit1, Rit2, Rem1, Rem2, Rad, Gem, Rheb1, Rheb2, Noey2, R-Ras, Rerg, RalA, RalB, RasD1, RasD2, RRP22, RasL10B, RasL11A, RasL11B, Ris/RasL12, and FLJ22655. In one particular embodiment, said method detects a mutation in codon 12 or 13 of KRas gene. Still in another embodiment, said method detects a mutation in codon 12 of KRas gene.

As stated herein, methods and apparatuses of the invention utilize a sample taken from a subject without labeling or amplication. Thus, methods and apparatuses of the invention avoid possible source of errors introduced by labeling or during amplification. In some embodiments, the specificity of said method is at least about 90%, typically at least about 95%, often at least about 98%, and most often at least about 99%. Still in other embodiments, the sensitivity of said method is at least about 90%, typically at least about 95%, often at least about 98%, and most often at least about 99%.

Yet in other embodiments, methods of the invention are capable of detecting mutation that is present in 0.1% or less, typically 0.05% or less, often 0.01% or less, and most often 0.001% or less in said sample.

Still in other embodiments, said sample is used to detect the presence of gene mutation without amplification, labeling, or modification. Yet in other embodiments, said sample is used to detect the presence of gene mutation without amplification or labeling.

Yet another aspect of the invention provides a method of diagnosing for a presence of cancer in a subject, said method comprising:
contacting a fluid sample obtained from subject with a solid substrate comprising a probe oligonucleotide under conditions sufficient to form a target-probe oligonucleotide duplex when a target oligonucleotide is present in said sample, wherein said probe oligonucleotide comprises at least a portion of a wild-type Ras gene; and
analyzing said target-probe oligonucleotide duplex for the presence of a mismatched target-probe oligonucleotide duplex with an atomic force microscope (AFM) comprising a DNA mismatch repair protein attached to a cantilever of said AFM,
   wherein the presence of said DNA mismatched target-probe oligonucleotide duplex is an indication that the subject has cancer.

In some embodiments, said wild-type Ras gene comprises a wild-type KRas gene. In one particular embodiment, said method is used to determine the presence of a mutation in codon 12 or 13 of KRas gene. Yet in another particular embodiment, said method is used to determine the presence of G12D, G12A, G12R, G12C, G12S, G12V, G13D, or a combination thereof.

Still in other embodiments, said step of contacting said fluid sample with said solid substrate further comprises the step of contacting said fluid sample with a blocking probe under conditions sufficient to selectively form a blocking probe-mutated Ras gene duplex and a single stranded mutated Ras gene, when said mutated Ras gene is present in said fluid sample. In one particular embodiment, said blocking probe comprises locked-nucleic acid/DNA (“LNA/DNA”) chimeric blocking probe. Still in another particular embodiment, the melting temperature difference between the LNA/DNA chimeric blocking probe-normal Ras gene duplex and a LNA/DNA chimeric blocking probe-mutated Ras gene duplex is at least about 5° C., typically at least about 10° C., and often greater than 10° C. In one particular embodiment, the melting temperature difference between the LNA/DNA chimeric blocking probe-normal Ras gene duplex and a LNA/DNA chimeric blocking probe-mutated Ras gene duplex is great than 12° C.

Further aspects of the invention provide an apparatus for detecting a gene mutation in a subject, said apparatus comprising:
a solid substrate comprising a probe oligonucleotide comprising at least a portion of a wild-type gene of interest; and
(ii) an atomic force microscope (“AFM”) comprising a DNA mismatch repair protein attached to a cantilever tip of said AFM.

In some embodiments, said probe oligonucleotide comprises an oligonucleotide for detecting a mutation in codon 12 or 13 of KRas gene. Still in other embodiments, said probe oligonucleotide comprises from about 10 to about 500 nucleotides, typically from about 20 to about 250 nucleotides, often from about 20 to about 200 nucleotides, and most often from about 25 to about 100 nucleotides. In one particular embodiment, said probe oligonucleotide comprises from about 20 to 250 nucleotides.

Another aspect of the invention provides a method for directly diagnosing a gene mutation in a subject. The method includes:
contacting a sample obtained from a subject with a solid substrate comprising a probe oligonucleotide under conditions sufficient to form a target-probe oligonucleotide duplex when a target oligonucleotide is present in said sample, wherein said probe oligonucleotide comprises a complementary oligonucleotide sequence of a wild-type gene;
measuring a level of interaction between said target-probe oligonucleotide duplex and a DNA mismatch repair protein using an atomic force microscope (AFM); and
analyzing said level of interaction to determine the presence of a mismatched target-probe oligonucleotide duplex,
   wherein the presence of mismatched target-probe oligonucleotide duplex is indication of a presence of a gene mutation in said subject.

In some embodiments, said probe oligonucleotide comprises a complementary oligonucleotide of a wild-type gene selected from the group consisting of Ras, BRAF, EGFR, RET, and PIK3CA. In some instances, said Ras gene is selected from the group consisting of KRas, HRas, NRas, R-Ras, M-Ras, E-ras, Di-Ras1, Di-Ras2, NKIRas1, NKIRas2, TC21, Rap1, Rap2, Rit1, Rit2, Rem1, Rem2, Rad, Gem, Rheb1, Rheb2, Noey2, R-Ras, Rerg, RalA, RalB, RasD1, RasD2, RRP22, RasL10B, RasL11A, RasL11B, Ris/RasL12, and FLJ22655. Yet in other instances, the method detects a mutation in codon 12 or 13 of KRas gene. In one particular instance, the wild-type gene comprises BRAF. In particular, said method detects a V600E mutation. Still in another particular instance, said method detects a M918T mutation. It should be appreciated that when detecting a specific mutation, the probe oligonucleotide comprises the particular location of the mutation to be detected. For example, when detecting V600E mutation, with a probe oligonucleotide having 25 nucleic acids, the probe-oligonucleotide may start at BRAF nucleic acid anywhere from 576 to 600 and end at 600 to 624, respectively. In this manner, all possible nucleic acid sequences of BRAF are within the scope of the disclosure that includes nucleic acid sequence 600 of BRAF. For example, the probe oligonucleotide may be oligonucleotide having nucleic acid sequence of BRAF 576-600, 577-601, 578-603, . . . , 600-624. In a similar manner, if the probe oligonucleotide has 100 nucleic acid sequences, the probe oligonucleotide may be oligonucleotide having nucleic acid sequence of BRAF 501-600, 502-601, 503-602, . . . , 600-699. In a similar manner, when detecting RET M918T mutation, the probe oligonucleotide includes amino acid sequence 918 of RET gene.

Yet in other embodiments, said sample comprises cell free DNA (cfDNA).

In further embodiments, said step of contacting said sample with said solid substrate further comprises the step of contacting said sample with a blocking probe under conditions sufficient to selectively form a blocking probe-mutated gene duplex and a single stranded mutated gene, when said mutated gene is present in said fluid sample. In some instances, said blocking probe comprises locked-nucleic acid/DNA (“LNA/DNA”) chimeric blocking probe. Yet in other instances, a melting temperature difference between a LNA/DNA chimeric blocking probe-normal gene duplex and a LNA/DNA chimeric blocking probe-mutated gene duplex is at least 10° C.

Still another aspect of the invention provides a method of directly diagnosing for a presence of cancer in a subject. The method typically includes:
contacting a fluid sample obtained from subject with a solid substrate comprising a probe oligonucleotide under conditions sufficient to form a target-probe oligonucleotide duplex when a target oligonucleotide is present in said sample, wherein said probe oligonucleotide comprises at least a portion of a wild-type Ras gene; and
analyzing said target-probe oligonucleotide duplex for the presence of a mismatched target-probe oligonucleotide duplex with an atomic force microscope (AFM) comprising a DNA mismatch repair protein attached to a cantilever of said AFM,
   wherein the presence of said DNA mismatched target-probe oligonucleotide duplex is an indication that the subject has cancer.

In some embodiments, said wild-type Ras gene comprises a wild-type KRas gene. In some instances, the method is used to determine the presence of a mutation in codon 12 or 13 of KRas gene. In some particular instances, the method is used to determine the presence of G12D, G12A, G12R, G12C, G12S, G12V, G13D, or a combination thereof.

Yet in other embodiments, said step of contacting said fluid sample with said solid substrate further comprises the step of contacting said fluid sample with a blocking probe under conditions sufficient to selectively form a blocking probe-mutated Ras gene duplex and a single stranded mutated Ras gene, when said mutated Ras gene is present in said fluid sample. In some instances, said blocking probe comprises locked-nucleic acid/DNA (“LNA/DNA”) chimeric blocking probe. Still in other instances, a melting temperature difference between a LNA/DNA chimeric blocking probe-normal Ras gene duplex and a LNA/DNA chimeric blocking probe-mutated Ras gene duplex is at least 10° C.

Still in other embodiments, said fluid sample comprises a cell free DNA (cfDNA).

One particular aspect of the disclosure provides a method for directly screening a subject for cancer, said method comprising:
analyzing a cell free DNA (cfDNA) obtained from subject with a solid substrate comprising a probe oligonucleotide under conditions sufficient to form a target-probe oligonucleotide duplex when a target oligonucleotide is present in said cfDNA, wherein said probe oligonucleotide comprises at least a portion of a wild-type BRAF gene or a wild-type Ras gene; and
analyzing said target-probe oligonucleotide duplex for the presence of a mismatched target-probe oligonucleotide duplex with an atomic force microscope (AFM) comprising a DNA mismatch repair protein attached to a cantilever of said AFM, wherein the presence of said DNA mismatched target-probe oligonucleotide duplex is used to screen the subject for presence of cancer.

In some embodiments, said cancer comprises thyroid cancer.

Still in other embodiments, said method is used to detect V600E mutation.

### BRIEF DESCRIPTION OF THE DRAWINGS

FIG. 1 is a schematic illustration of KRAS mutation detection by tracking the single-molecular adhesion event of a MutS-tethered AFM tip. The target molecules from the sample solution were hybridized to the surface-immobilized capture probe. The wild-type (WT) target molecule formed a fully matched duplex upon hybridization, while the mutated target molecule formed a singly mismatched duplex upon binding to the capture probe. MutS can only bind to the mismatched duplex and generated the specific force-distance curves upon unbinding, whereas the fully matched duplex remained silent to MutS.

FIGS. 2A-2C show localization of an individual surface-captured mutated KRAS G12D gene via high-resolution QI mapping. FIG. 2A is schematic illustration of measurement of cluster radius through the detection of MutS binding to the surface-captured mismatched DNA duplex. The MutS-modified AFM tip scanned the surface with a 5 nm pixel size, and a cluster of specific pixels was observed. FIG. 2B is histograms of the adhesion force values (top) and stretching distance (bottom) measured from the specific FD curves. FIG. 2C shows adhesion force map (top) and the corresponding ellipse fitting image (bottom) of a representative cluster.

FIG. 3A is a schematic representation of superimposing the successive specific adhesion maps to produce final overlaid map at a particular location.

FIG. 3B shows representative overlaid adhesion force maps obtained during the quantification of KRAS G12D mutated DNA with 0.1% allele frequency in cfDNA sample for sample volume of 1.0 μL copies (300×300 pixels, 3.0×3.0 μm2).

FIG. 3C shows representative overlaid adhesion force maps obtained during the quantification of KRAS G12D mutated DNA with 0.1% allele frequency in cfDNA sample for sample volume of 0.6 μL copies (200×200 pixels, 2.0×2.0 μm2). The white dashed circles mark the boundary of the respective capture probe spot corresponding to morphology map. The qualified clusters are marked with solid white circles.

FIG. 4 is a schematic illustration showing generation of the desired target molecule in the single-stranded form via effective blocking with LNA/DNA chimeric blocking probe during annealing (steps A-C). The melting temperature of the duplex formed by involving the blocking probe was 95.2° C., whereas that for the 156-mer DNA duplex was 85.5° C.

FIG. 5 is a schematic representation of the immobilization of a his-tagged MutS protein on a Ni-NTA modified AFM tip.

FIGS. 6A-6D are histograms of most probable unbinding force for the interaction between MutS-protein-modified AFM tip and a DNA duplex containing (FIG. 6A) triple base mismatches and (FIG. 6B) triple base deletions. Histograms were constructed from the specific force distance curves. No specific adhesion event was observed for (FIG. 6C) five base mismatch and (FIG. 6D) five base deletion.

FIG. 7 (Panels A-C) shows results of control experiments to assess the reliability of the interaction between MutS-protein-modified AFM tip and on-surface singly mismatched DNA duplex. The on-surface mismatched DNA duplex was generated via the hybridization of KRAS G12D mutated target DNA with the surface-immobilized capture DNA probe. (Panel A) Overlaid adhesion map (200×200 pixels, 2.0×2.0 μm²) obtained after hybridization (900 zM, 40 μL) with WT KRAS DNA. (Panel B) Overlaid adhesion map (200×200 pixels, 2.0×2.0 μm²) resulting from the hybridization (900 zM, 40 μL) of LNA/DNA blocking probe. (Panel C) Overlaid adhesion map for the DNA capture probe spot (200×200 pixels, 2.0×2.0 μm²) without target hybridization. No positive cluster was observed in all cases.

FIG. 8A is a schematic illustration of methods to confirm the presence of any type of KRAS mutation in a cfDNA sample.

FIG. 8B is a schematic illustration of methods for identifying KRAS mutation type.

FIG. 9 is a plot showing the correlation between the number of KRAS G12D mutant present in a sample solution (40 μL) and the number of positive cluster observed. A linear fitting was performed with the data, and the slope was 0.63 with an adjusted R² value of 0.995.

### DETAILED DESCRIPTION OF THE INVENTION

Methods and apparatuses of the invention can be used in a wide variety of clinical diagnostic applications including, but not limited to, detection and/or analysis of biomarkers related to, but not limited to, cancer, trauma, sepsis, aseptic inflammation, myocardial infarction, stroke, transplantation, diabetes, sickle cell disease, pregnancy, fetal genetic anomaly determination, as well as other clinical conditions in which an oligonucletodie biomarker can be obtained from a subject's biological sample (or simply “sample”). Exemplary biological samples that can used in methods and apparatuses of the invention include, blood, plasma, saliva, mucous, stool, urine, tear, cells, tissues, ascites, pleural effusion, sputum, cerebrospinal fluid (CSF), lymph, as well as any other material obtained from a subject that contains an oligonucleotide or DNA or a fragment thereof.

In clinical diagnostics, early detection of a disease is most effective in intervention and treatment. This is particularly true for cancer treatment. While many therapies are available in treating various cancers, in oncology early detection and intervention is still considered to be the most effective solution to reduce cancer mortality. Therefore, the role of sensitive and specific detection/quantification of relevant biomarkers for a number of clinical conditions at an early stage is important in terms of survivability as well as successful treatment. This is particularly true for cancers in which early symptoms are not evident or for which a relatively simple and noninvasive technique, such as mammography, is not applicable. cfDNA has been shown to be a useful biomarker for a multitude of clinical conditions including, but not limited to, cancer, fetal medicine, trauma, sepsis, aseptic inflammation, myocardial infarction, stroke, transplantation, diabetes, sickle cell disease, as well as other clinical conditions associated with the presence of cfDNA and/or gene mutations.

For the sake of clarity, brevity, convenience, and illustration, the present invention will now be described in reference to diagnosis of cancer. However, it is to be understood that the invention as a whole is not intended to be so limited, and that one skilled in the art will readily recognize that the concept of the invention will be applicable to other clinical diagnosis, monitoring therapeutic efficacy of a particular treatment, and/or determining a therapeutic protocol for a particular clinical condition. These other clinical conditions, treatment, and diagnosis include fetal medicine, trauma, sepsis, aseptic inflammation, myocardial infarction, stroke, transplantation, diabetes, sickle cell disease, as well as other clinical conditions associated with the presence of cfDNA and/or gene mutations. These and other clinical conditions suitable for use in the instant invention will be readily apparent to those skilled in the art.

Mutation of ras genes (H-ras, N-ras, and K-ras) is commonly associated with many tumor types and has been implicated in the development of cancers. In this regard, the KRAS mutation is particularly important because studies have shown that it is observed in 90% of pancreatic cancer and in 30-60% of colon cancer. The KRAS mutation is located in codon 12 or codon 13 of exon 2 and is often considered the most frequently detected activating mutation in human cancers. In addition, KRAS mutation has been observed in the early stages of pancreatic and colorectal cancers. Some of the specific KRAS mutations known to date include, but are not limited to:

| Codon | Base Pair Substitution | Amino Acid Change |
|---|---|---|
| 12 | GGT → GCT | Gly → Ala (G12A) |
| 12 | GGT → GAT | Gly → Asp (G12D) |
| 12 | GGT → CGT | Gly → Arg (G12R) |
| 12 | GGT → TGT | Gly → Cys (G12C) |
| 12 | GGT → AGT | Gly → Ser (G12S) |
| 12 | GGT → GTT | Gly → Val (G12V) |
| 13 | GGC → GAC | Gly → Asp (G13D) |

Currently, tissue biopsy is the gold standard for diagnosing KRAS mutation. However, the standard biopsy technique suffers from certain disadvantages. As tumors and metastases are not always accessible for biopsy, sample collection often requires invasive procedures, and intratumoral heterogeneity is not well understood. An alternative technique, called ‘liquid biopsy’, has been introduced to overcome some of these limitations. In this technique, genomic alterations (somatic mutations) in solid cancerous tumors are characterized by analyzing the presence of circulating cell-free tumor DNA (ctDNA) in the blood or other body fluids, such as saliva, urine, ascites, and pleural effusion.

Without being bound by any theory, it is believed that in general ctDNAs are released into the bloodstream via necrosis, autophagy, apoptosis, and other physiological events induced by, e.g., microenvironmental stress. For a wide range of malignancies, a consistent correlation has been established between the primary tumor and the respective ctDNA. Hence, ctDNA analysis can be useful for early diagnosis of cancer, residual disease monitoring, and tracking individual responses to the employed therapy. Furthermore, the ctDNA concentration might offer prognostic insights, as an enhanced ctDNA concentration has been often associated with tumor progression and reduced survival. Several methods to detect and quantify ctDNA related to KRAS mutation, such as quantitative real-time PCR (qPCR) using ARMS primers, mutant-enriched PCR, COLD-PCR, digital PCR, Sanger sequencing, and next generation sequencing (NGS) have been reported. However, the limit of detection (LOD) and sensitivity/specificity of these techniques are not sufficient, as most early stage solid tumors exhibit very low levels of ctDNA. It has been shown that the LOD for the standard sequencing method is approximately 20% mutant alleles, whereas for NGS, it has been shown that it can reach as low as approximately 2-6%. An LOD of approximately 1% has been observed for the ARMS-PCR method, while mutant-enriched PCR and COLD-PCR have greater sensitivity for detecting KRAS mutations, with an LOD of approximately 0.1%. Recently, LODs of 0.05% and 0.01% have been reported for chip-based dPCR and droplet-based dPCR, respectively, for KRAS mutation.

However, achieving greater sensitivity without compromising the specificity of the measurement remains a challenge for PCR-based methods. The terms “sensitivity” and “specificity” are used herein in their conventional well recognized meaning by one skilled in the art. Briefly, these terms relate to statistical measures of correctly identifying actual positives (“sensitivity”) and actual negatives (“specificity”). Thus, in general, a 100% sensitive test or diagnostic method will correctly identify all positives, and a 100% specificity test or diagnostic method will correctly identify all negatives. Many factors can affect the specificity of a PCR, such as primer sequence and purity, template DNA purity, annealing temperature, Mg²⁺ ion concentration and other additives, such as dimethyl sulfoxide (DMSO), glycerin, betaine and formamide, which are commonly found in PCR mixtures. In particular, the specificity of a PCR is greatly affected for samples with very low template DNA concentrations. Accordingly, any method involving a PCR will inherently reduce the sensitivity and/or specificity.

In one aspect, methods of the invention involve a direct quantification without a need for any amplification, labelling, or modification of the sample, thereby significantly increasing the sensitivity and/or specificity. As used herein, the term “modification” when referring to a sample preparation means changes to the sample through a synthetic chemical reaction to produce a product that is different from its natural state present in the sample. Thus, in some aspects, unlike most conventional diagnostic tests, methods of the invention do not require or involve subjecting the sample to a chemical reaction to change the molecule to a different product for testing. It should be appreciated, however, that annealing a single stranded oligonucleotide to form a double stranded oligonucleotide or denaturing a double stranded oligonucleotide to form a single stranded oligonucleotide is not within the definition of “modification” as these processes do not change any of the amino acids. Annealing and denaturing merely involves forming a complex or a single stranded form, respectively, of the same chemical entity.

Thus, in one particular aspect of the invention, a method is provided involving a direct quantification approach/technique without amplification. Methods of the invention can achieve single-molecule detectability in combination with high sensitivity/specificity, thereby overcoming many limitations present in conventional diagnostic methods such as ones involving a PCR amplification.

In some aspects, methods of the invention include atomic force microscopy (AFM)-based single-molecule force spectroscopy. AFM-based methods of the invention allow for the probing of intra- and intermolecular forces with sensitive responses under physiological conditions without any labeling or amplification. The present inventors have previously shown that by exploiting the force-volume mode of AFM, a translocated gene of 1-10 copies can be quantified directly in the presence of a normal gene of a million copies.

As an illustration of methods and apparatuses of the invention, the invention will now be described in reference to directly (i.e., without amplification or labeling) detecting KRAS G12D mutation in cfDNA samples with very low mutant allele frequencies (0.1%) at high sensitivity/specificity (near 100%) by force-distance (F-D) curve-based AFM.

Some aspects of the invention include use of a DNA mismatch repair protein for recognizing the presence of a mismatched DNA duplexes in lieu of fully matched DNA duplex. Unless context requires otherwise, the terms “oligonucleotide” and “DNA” are used interchangeably herein to refer to a polynucleotide whose molecules contain a relatively small number of nucleotides. In generally, oligonucleotides in a subject's sample or oligonucleotides used in methods and apparatuses of the invention contain from about 10 to about 500 nucleotides, typically from about 20 to about 250 nucleotides, often from about 20 to about 200 nucleotides, and most often from about 25 to about 150 nucleotides. In one particular embodiment, said probe oligonucleotide comprises from about 20 to 250 nucleotides. Oligonucleotides can be naturally occurring (e.g., cfDNAs) or it can be synthetically manufactured or produced (e.g., a probe oligonucleotide). Moreover, unless context requires otherwise, the term “DNA” may refer to a duplex form or a single stranded form (e.g., after denaturization). DNA mismatch repair proteins are well known to one skilled in the art and can be obtained from prokaryote cells (e.g., E. coli, tag) or eukaryote cells (e.g., hMSH). Exemplary DNA mismatch repair proteins useful in methods and apparatuses of the invention include, but are not limited to, MutS protein complex (“MutS”) and MSH protein complex (“MSH”) (e.g., MSH2-MSH6 (MutS alpha) complex, MSH2-MSH3 (MutS beta) complex)

In one particular embodiment of the invention, MutS protein, which binds to only the mismatched DNA duplexes in lieu of fully matched ones, was utilized to detect the presence of a mutated gene in a sample. In one particular embodiment, samples used in methods and apparatuses of the invention include any fluid sample obtained from a subject that may include cfDNAs. Exemplary samples that can be used in the invention include, but are not limited to, blood and other body fluids, such as saliva, urine, ascites, pleural effusion, sputum, cerebrospinal fluid (CSF), lymph, and stool. The term “subject” refers to mammals such as equines, bovines, felines, canines, Sus, primates, Homo sapiens, etc. Typically, the subject is human or a domesticated animal.

One specific aspect of the invention provides a method for detecting a presence of a mutated gene in a sample. The method includes:
contacting the sample comprising a target oligonucleotide with a solid substrate to which is attached a probe oligonucleotide under conditions sufficient to form a target-probe oligonucleotide duplex; and
analyzing the target-probe oligonucleotide duplex for the presence of a mismatched target-probe oligonucleotide complex with an atomic force microscope (AFM) having an AFM tip comprising a DNA mismatch repair protein.
   The presence of the mismatched target-probe oligonucleotide duplex is an indication that a mutated gene is present in said sample. The probe oligonucleotide comprises a complementary oligonucleotide sequence of a normal or a wild-type gene such that a duplex can be formed if the target gene is present in the sample. Unless the context requires otherwise, any reference to a probe oligonucleotide or a target DNA or target oligonucleotide refers to a single stranded oligonucleotide or DNA.

It should be appreciated when using a sample that may contain cfDNA(s), the sample may be subjected to a denaturing condition so that a single stranded cfDNA is formed. The single stranded cfDNA is then allowed to bind to the probe oligonucleotide to form a target-probe oligonucleotide complex. The AFM is then used to determine whether this target-probe oligonucleotide complex (i.e., duplex) includes any mismatched target-probe oligonucleotide complex. It should also be appreciated that by selecting an appropriate probe oligonucleotide, one can analyze a presence of mutated gene for a wide variety of genes of interest. For example, by selecting a wild-type of an oncogene (i.e., a gene that has the potential to cause cancer) as the probe oligonucleotide, one can analyze the sample for a possible presence of cancer in a subject. The terms “normal” and “wild type” are used interchangeably herein and refers to “a phenotype, genotype, or gene that predominates in a natural population of organisms or strain of organisms” where no clinical condition or disease is expressed. As will be recognized, the presence of a mutant gene does not necessarily indicate that the subject is suffering from cancer (or other clinical conditions as determined by the probe oligonucleotide). For example, the presence of a mutant (i.e., a particular allele of) BRAC1 and BRAC2 genes is not indicative that the subject has a breast cancer, but it merely indicates that the subject is more prone to onset of breast cancer relative to a subject having a wild type BRAC1 and/or BRAC2 genes.

For diagnosis of a possible presence of cancer, in some embodiments the probe oligonucleotide comprises a complementary oligonucleotide of a wild type gene selected from the group consisting of Ras, EGFR, and PIK3CA. In some instances, the Ras gene is selected from the group consisting of KRas, HRas, NRas, R-Ras, M-Ras, E-ras, Di-Ras1, Di-Ras2, NKIRas1, NKIRas2, TC21, Rap1, Rap2, Rit1, Rit2, Rem1, Rem2, Rad, Gem, Rheb1, Rheb2, Noey2, R-Ras, Rerg, RalA, RalB, RasD1, RasD2, RRP22, RasL10B, RasL11A, RasL11B, Ris/RasL12, and FLJ22655.

Still in other embodiments, the method of the invention is used to detect a mutation in codon 12 or 13 of KRas gene. In one particular embodiment, the method is used to detect a mutation in codon 12 of KRas gene. One specific embodiment of the invention detects the presence of KRAS G12D mutation.

Yet in other embodiments, the specificity of methods of the invention is at least about 90%, typically, at least about 95%, often at least about 97%, still more often at least about 98%, yet more often at least about 99% still yet more often at least about 99.5%, and most often at least about 99.8%. When referring to a numerical value, the terms “about” and “approximately” are used interchangeably herein and refer to being within an acceptable error range for the particular value as determined by one of ordinary skill in the art, which will depend in part on how the value is measured or determined, e.g., the limitations of the measurement system, i.e., the degree of precision required for a particular purpose. For example, the term “about” typically means within 1 standard deviation, per the practice in the art. Alternatively, the term “about” can mean±20%, typically ±10%, often ±5% and more often ±1% of the numerical value. In general, however, where particular values are described in the application and claims, unless otherwise stated, the term “about” means within an acceptable error range for the particular value.

In further embodiments, the sensitivity of methods of the invention is at least about 90%, typically, at least about 95%, often at least about 97%, still more often at least about 98%, yet more often at least about 99% still yet more often at least about 99.5%, and most often at least about 99.8%

Still in other embodiments, the methods of the invention are capable of detecting mutation that is present in about 10% or less, typically about 5% or less, often about 1% or less, more often about 0.1% or less, and most often about 0.01% or less in the sample.

As stated herein, methods and the apparatuses of the invention can be used without amplification, labeling, or modification of the sample. By eliminating a need for amplification, labeling or modification, the specificity and/or selectivity of the invention are significantly higher than those of any conventional methods. Furthermore, because of the ability of methods and apparatuses of the invention to utilize a minute quantity of sample for analysis, the labor time and cost are also significantly reduced. In some embodiments, the amount of sample required in methods and apparatuses of the invention is no more than about 10 mL, typically no more than about 1 mL, often no more than about 0.5 mL, more often no more than about 0.1 mL, and most often no more than about 0.05 mL.

The sensitivity and/or selectivity may depend on the stability of the target-probe oligonucleotide complex that is formed. In general, the higher number of hybridized base pairs will result in more stable target-probe oligonucleotide complex. Accordingly, in some embodiments, the probe oligonucleotide comprises from about 10 to about 100, typically from about 15 to about 80, often from about 20 to about 60, more often from about 25 to about 50, and most often from about 30 to about 40 nucleotides that are complementary to the normal target gene of interest. It should be appreciated that the probe oligonucleotide can include other nonbinding portions, such as polyT for attaching the probe oligonucleotide to the solid substrate surface. Thus, while the total number of nucleotides can be relatively large, e.g., greater than 100, the number of nucleotides referred to herein is in reference to nucleotides that are designed for complementary binding to the normal target gene of interest.

Circulating free DNA (cfDNA) are degraded DNA fragments released to the blood plasma and generally consists of small fragments of DNAs. cfDNA is mostly a double-stranded extracellular molecule of DNA, consisting of small fragments (e.g., from about 70 to about 200 bp) as well as larger fragments. Some ctDNAs have been recognized as particularly useful and accurate marker for the diagnosis of cancers, such as colon cancer, prostate cancer and breast cancer. Accordingly, in some embodiments of the invention methods and apparatuses are used to diagnose the presence of colon cancer, prostate cancer, breast cancer, pancreatic cancer, lung cancer, melanoma, and bladder cancer, as well as other solid tumors and cancers.

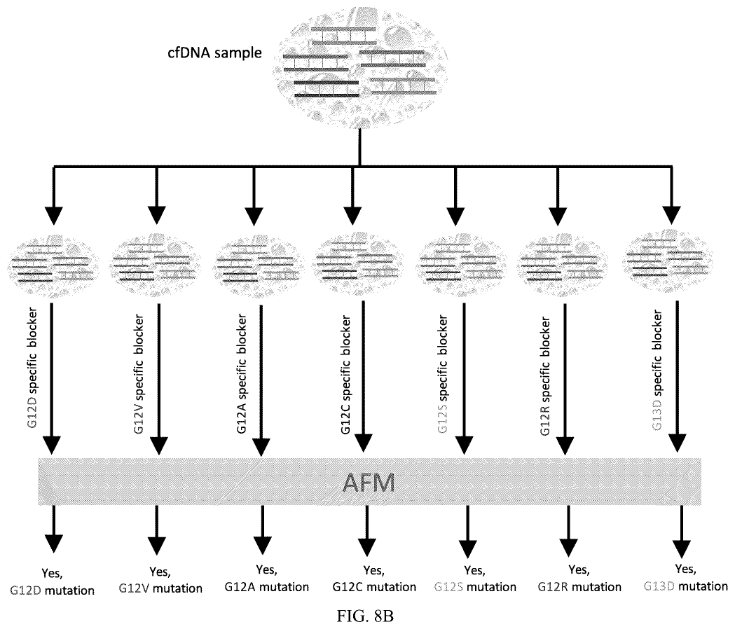

Yet in other embodiments, methods and the apparatuses of the invention can. be used to identify the mutation in the target gene. Such methods and apparatuses can also include use of a locked nucleic acid/DNA (“LNA/DNA”) chimeric blocking probe as discussed herein and as schematically illustrated in FIGS. 8A and 8B. In particular, by using LNA/DNA chimeric blocking probe(s), one can produce a mismatched target-probe oligonucleotide complex for a single allele (i.e., mutant gene) to be detected by the DNA mismatch repair protein. In this manner, one can readily identify the allele of a gene that is present in the sample.

The DNA mismatch repair protein can be attached to the AFM tip by any of the methods known to one skilled in the art. In one particular embodiment, the DNA mismatch repair protein is histidine-tagged. This allows attachment of the DNA mismatch repair protein by forming a complex with a Ni(ii) ion that is present or attached to the AFM tip. Furthermore, by chelating the histidine-tagged DNA using a Ni(II) ion complex, one can readily replace the DNA mismatch repair proteins as desired. One particular embodiment of attaching the DNA mismatch repair protein is exemplified in the Examples section where Ni(II) ion complex and a dendron is used.

As stated throughout this disclosure, in some embodiments, LNA/DNA chimeric blocking probe can be added to the mixture of the sample and the solid substrate. In this manner, one or more particular alleles of the gene is allowed to bind to the LNA/DNA chimeric blocking probe to increase the presence of its/their complementary single stranded oligonucleotides to bind to the probe oligonucleotide.

LNA/DNA chimeric blocking probe are introduced into the sample to increase duplex stability and specificity of one or more alleles of the target DNA. For example, by forming a stable duplex with a minor allele in the sample, one can increase the relative concentration of the single stranded DNA of the minor allele. In this manner, formation of a duplex between the single stranded DNA of the minor allele and the probe oligonucleotide is significantly increased relative to wild type or other alleles. In some embodiments, the amount of LNA/DNA chimeric blocking probe oligonucleotide used ranges from about 1 equiv. to about 100 equiv., typically from about 2 equiv. to about 20 equiv., more often from about 2 equiv. to about 10 equiv., and most often from about 2 or 3 equiv. relative to the theoretical amount of a desired minor allele present in the sample for detection.

The length of LNA/DNA chimeric blocking probe typically ranges from about 10% to about 100%, often from about 20% to about 80%, and most often from about 30% to about 50% of the length of the minor DNA allele to be detected. Alternatively, the length of LNA/DNA chimeric blocking probe typically ranges from about 10% to about 100%, often from about 25% to about 100%, more often from about 50% to about 100% of the length of the probe oligonucleotide that is complementary to the normal target gene for detection. Still alternatively, the LNA/DNA chimeric blocking probe has from about 10 to about 100, typically from about 15 to about 80, often from about 20 to about 60, more often from about 25 to about 50, and most often from about 30 to about 40 nucleotides. As is well known to one skilled in the art, the amount of locking riboside used will influence the stability of the LNA/DNA chimeric blocking probe-minor DNA allele complex. In some embodiments, at least about 20%, typically at least about 40%, often at least about 60%, more often at least about 80%, and most often about 100% of the nucleotides of the LNA/DNA chimeric blocking probe is locked nucleotide. Alternatively, the melting temperature of LNA/DNA chimeric blocking probe-minor DNA allele complex is increased by at least about 10° C., typically by at least about 14° C., often by at least about 16° C., more often by at least about 20° C., and most often by at least 23° C. Still in another embodiment, the melting temperature of LNA/DNA chimeric blocking probe-minor allele gene duplex is at least about 10° C., typically at least about 12° C., often at least about 15° C., and most often at least about 21° C. higher than the melting temperature of a LNA/DNA chimeric blocking probe-normal gene duplex.

Another aspect of the invention provides an apparatus for detecting a gene mutation in a subject. The apparatus includes:
a solid substrate comprising a probe oligonucleotide of a normal gene of interest; and
(ii) an atomic force microscope (“AFM”) having an AFM tip (i.e., cantilever tip) comprising a DNA mismatch repair protein.

Still another aspect of the invention provides an atomic force microscope having a cantilever tip comprising a DNA mismatch repair protein.

Use of a DNA mismatched repair protein to detect the presence of a mismatched target-probe oligonucleotide complex allows AFM to sense or detect as well as quantify only the mutated genes or any target-probe oligonucleotide that is mismatched. As stated herein, in some embodiments a specifically designed LNA/DNA chimeric blocking probe is used with a sample comprising cfDNA prior to the denaturation step to make the desired target sequence accessible to the capture probe in the single-stranded form.

Efficient scanning of the capture spot area can be ensured fabricating miniaturized capture probe spots. Such use of defined capture probe spot provides a significantly lower LOD compared to conventional methods.

The solid substrate and/or the AFM tip can additionally include dendron in order to provide further sensitivity and/or specificity. In some embodiments, dendron is those disclosed in a commonly assigned U.S. Pat. No. 9,671,396, issued Jun. 6, 2017, which is incorporated herein by reference in its entirety. Briefly, such a dendron compound is of the formula:

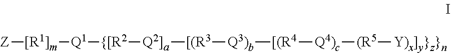

where
each of m, a, b, and c is independently 0 or 1;
x is 1 when c is 0 or when c is 1, x is an integer from 1 to the oxidation state of Q⁴-1;
y is 1 when b is 0 or when b is 1, y is an integer from 1 to the oxidation state of Q³-1;
z is 1 when a is 0 or when a is 1, z is an integer from 1 to the oxidation state of Q²-1;
n is an integer from 1 to the oxidation state of Q¹-1;
Q¹ is a central atom having the oxidation state of at least 3;
each of Q², Q³ and Q⁴ is independently a branch atom having the oxidation state of at least 3;
each of R¹, R², R³, R⁴, and R⁵ is independently a linker;
Z is the functional group that is attached to the probe oligonucleotide (in case of a solid substrate) or the DNA mismatched repair protein (in case of an AFM tip); and
each of Y is independently a functional group on the terminus of said base portion,
   wherein a plurality of Y are attached to said first surface of said solid support, provided the product of n, x, y, and z is at least 3.

In some embodiments, the product of n, x, y, and z is 9, or 27.

It should be appreciated that Z can optionally include other linker(s) such as polyT oligonucleotides, polyethylene glycol (“PEG”) linker etc. In one particular embodiment, the AFM tip includes a linker with a chelating group that is used to chelate a Ni(II) ion such that a histidine-tagged DNA mismatched repair protein is attached. Still in other instances, Z comprises a heteroatom selected from the group consisting of N, O, S, P, and a combination thereof.

One particular aspect of the disclosure is directed to detecting V600E mutation, i.e., a mutation of the BRAF gene in which valine (V) is substituted by glutamic acid (E) at amino acid 600. This V600E mutation has been shown to be associated with melanoma, hairy cell leukemia, papillary thyroid carcinoma, colorectal cancer, non-small-cell lung cancer, Langerhans cell histiocytosis, Erdheim-Chester disease (a non-Langerhans-cell histiocytosis) and ameloblastoma. In one particular embodiment, methods of the disclosure are used to directly detect (i.e., without any amplification, labeling, or modification of the target gene) a presence of V600E mutation from cfDNA of a subject. It should be appreciated that the term “directly detect” does not exclude manipulation of the sample to isolate, purify, or otherwise prepare a sample for analysis. The term only excludes amplification (e.g., via a PCR or other methods known to one skilled in the art), modification of the target gene to produce a different molecule, e.g., by attaching a label, such as fluoro-probes, radionuclides, or other detectable label moieties. In one particular instance, methods of the disclosure are used to detect BRAF V600E from cfDNA samples of cancer patients who underwent thyroidectomy. Regular examination of the samples for BRAF V600E allows monitoring of recurrence of thyroid cancer after the surgery. It should be appreciated, however, methods for detecting BRAF V600E from cfDNA samples from a subject can be used to determine recurrence of any cancer that is associated with V600E mutation. In this manner, one can monitor subjects who have successfully undergone a cancer treatment for any possible recurrence of cancer that is associated with V600E mutation.

Another particular example of use of methods of the disclosure is detection of mutation of the RET proto-oncogene, often simply referred to as RET. RET is a human gene that encodes instructions for making a receptor tyrosine kinase protein. Mutations in the RET gene have been implicated in various types of cancers including, but not limited to, medullary thyroid carcinoma (MTC), multiple endocrine neoplasias type 2A and 2B, pheochromocytoma, and parathyroid hyperplasia. Genetic testing for RET mutations is often used to help diagnose and determine the prognosis of certain cancers, particularly medullary thyroid carcinoma.

MTC is an aggressive form of thyroid cancer caused by mutations of the RET receptor tyrosine kinase (multiple endocrine neoplasia, type 2) or in the majority of sporadic MTC activating mutations of RET or RAS. RET mutations screening is a well-established test that allows for prophylactic surgery in asymptomatic gene carriers. In sporadic MTC, a single mutation, M918T, has been shown to account for the majority of RET mutations. The presence of somatic RET mutation, particularly M918T, has been associated with worse prognosis in some studies. Recent studies showed that patients with an RET M918T somatic mutation respond well to cabozantinib or vandetanib treatment.

While a direct somatic mutation testing is ideal to determine prognosis or diagnosis of MTC, for many patients somatic mutation testing of the primary tumor is not performed because of access to tumor tissue, or issues related to DNA amount or quality. Currently, diagnosis or prognosis test for MTC is based on the concentration of a protein biomarker, i.e., calcitonin. The level of calcitonin is also often used to determine recurrence of MTC after the surgery and vandetanib or cabozantinib treatment. In general, doubling of calcitonin concentration is indicative of the recurrence. Unfortunately, interpretation of calcitonin measurement to predict the prognosis of medullary thyroid carcinoma (MTC) requires multiple measurements over an extended time period, making it an imperfect biomarker for evaluating prognosis or disease behavior. Single circulating cell-free DNA (cfDNA) values have been shown to be a valuable prognostic marker for several solid tumors. Since it is expected that a successful surgery would lead to absence of any RET mutation, direct DNA biomarker test would lead to more accurate diagnosis and/or prognosis.

Some direct methods for determining the presence of DNA biomarker for MTC, namely, RET M918T mutation, has been studied using gene analytical approach, such as RT-PCR and droplet digital polymerase chain reaction (“ddPCR”). These gene analytical methods showed a high specificity for determining RET M918T mutation. Unfortunately, the sensitivity of these methods was low, thereby rendering amplification based gene analytical methods not suitable for determining the presence of RET M918T mutation in clinical applications.

As discussed herein, methods of the present disclosure have a high selectivity and high sensitivity. Accordingly, another particular aspect of the disclosure provides a method for detecting RET M918T mutation in a subject. In some embodiments, detection of RET M918T mutation is performed from cfDNA samples of a subject who underwent a prophylactic surgery in an asymptomatic gene carrier.

Methods of the disclosure are particularly useful as a noninvasive approach to identify tumoral mutation status is necessary when there is an absence of available tumor tissue or when metastatic tumor tissue is difficult to sample.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Preparation of capture probes: The DNA capture probe was designed to hybridize with both the WT KRAS sequence (fully complementary) and KRAS G12D mutated sequence (singly mismatched) at a comparable hybridization rate. A 96-mer custom-synthesized (Bioneer, Korea) DNA capture probe was employed in this study. Of 96 bases, 36 bases were available for target hybridization (Table 1), and the rest of the probe was a T₆₀ tail at the 3′-end. In addition, an amine group was introduced at the 3′-end of the capture probe for immobilization on a glass substrate. The target DNAs (156-mer) were custom synthesized (Integrated DNA Technologies Inc., USA) and consisted of the sequences of WT KRAS and KRAS G12D mutation (in codon 12), respectively.

A 96-mer custom-synthesized (Integrated DNA Technologies Inc., USA) DNA capture probe was used for the detection of EGFR L858R mutation (Table 1).

TABLE 1
Capture probes, WT sequences, KRAS G12D, EGFR L858R, and LNA/DNA chimeric
blockers used in this study. The underlined bases were hybridizable to
the capture probe. The LNA bases are marked with +A, +T, +G, +C.

| Name | Sequence |
|---|---|
| Capture probe (96-mer, | 5′-(Cy3)-GGC ACT CTT GCC TAC GCC ACC AGC TCC AAC TAC CAC |
| DNA) | TTT TTT TTT TTT TTT TTT TTT TTT TTT TTT TTT TTT TTT TTT |
| | TTT TTT TTT TTT TTT TTT-3′-C6-NH2 (SEQ ID NO: 1) |
| WT KRAS (156-mer, DNA, | 5′-ATA GTC ACA TTT TCA TTA TTT TTA TTA TAA GGC CTG CTG |
| Tₘ: 81.4° C. for duplex | AAA ATG ACT GAA TAT AAA CTT GTG GTA GTT GGA GCT GGT |
| fully matched with the | GGC GTA GGC AAG AGT GCC TTG ACG ATA CAG CTA ATT CAG |
| capture probe) | AAT CAT TTT GTG GAC GAA TAT GAT CCA ACA ATA GAG GTA-3′ |
| | (SEQ ID NO: 2) |
| KRAS G12D (156-mer, DNA, | 5′-ATA GTC ACA TTT TCA TTA TTT TTA TTA TAA GGC CTG CTG |
| Tₘ: 80.3° C. for duplex | AAA ATG ACT GAA TAT AAA CTT GTG GTA GTT GGA GCT GAT GGC |
| singly mismatched with | GTA GGC AAG AGT GCC TTG ACG ATA CAG CTA ATT CAG AAT CAT |
| the capture probe) | TTT GTG GAC GAA TAT GAT CCA ACA ATA GAG GTA-3′ |
| | (SEQ ID NO: 3) |
| LNA/DNA chimeric blocker | 5′-GTG G+TA G+T+T G+GA G+CT +G+A+T G+GC G+TA G+G+C |
| (36-mer) for KRAS G12D | A+AG A+GT GCC-3′ |
| | (SEQ ID NO: 4) |
| Capture probe for EGFR | 5′-GCA CCC AGC AGT TTG GCC AGC CCA AAA TCT GTG ATC TTT |
| | TTT TTT TTT TTT TTT TTT TTT TTT TTT TTT TTT TTT TTT TTT |
| | TTT TTT TTT TTT TTT-3′-3AmMO* (SEQ ID NO: 5) |
| LNA/DNA chimeric blocker | 5′- GAT C+AC A+G+A T+TT T+GG G+C+G +GG+C C+AA A+C+T |
| (36-mer) for EGFR L858R | G+CT G+GG TGC -3′ |
| | (SEQ ID NO: 5) |

Preparation of LNA/DNA chimeric blocking probes: Three 36-mer custom-synthesized (Exiqon, Denmark) chimeric blockers were employed for the detection of KRAS G12D and EGFR L858R mutations (Table 1).

Preparation of glass slides with wells: Glass slides were treated with inductively coupled plasma (ICP) to produce multiple wells at the National Institute for Nanomaterials Technology (NINT, Korea). Square wells of various sizes with a depth of 200 nm were produced on each slide. The slides were then coated with dendrons (27-acid dendrons) by NB POSTECH, Inc., followed by treatment with disuccinimidyl carbonate for activation.

Fabricating miniaturized capture probe spots onto etched glass slides: A cartridge kit with a premounted microchanneled cantilever, equipped with a pyramidal tip with a 300 nm aperture (Cytosurge AG, Switzerland), was employed to dispense the capture probe DNA solution onto the etched glass slides. The cantilever was 200 μm in length with a 1 μm microchannel and a typical spring constant of 2 N/m. A 20 μM capture probe solution was prepared in 2×SSC buffer (pH 8.5) (Sigma-Aldrich). Glycerol was added to the solution (12.5% v/v) to control the evaporation rate. The capture probe solution (8 μL) was then placed into the reservoir of a FluidFM, and the cantilever was mounted on an AFM (FlexAFM, Nanosurf, Switzerland) connected to a pressure controller (FluidFM microfluidics control system, Cytosurge AG). The cantilever was brought into contact with the surface, and an overpressure of +1000 mbar was applied for 1 min to fill the entire microchannel with the capture probe solution. A set point of 200 mV was applied during the approach step, and the spot size was controlled by adjusting two key parameters: applied pressure and contact time. Spotting was performed onto etched square wells of 20×20 μm², and the spot position (x and y coordinates) was recorded. After spotting, the slides were placed in a humidity chamber (80% humidity) at room temperature for 12 h. Next, the slides were washed with 2×SSC buffer (pH 7.4) (Sigma-Aldrich) containing 0.2% SDS at 40° C. for 20 min, followed by washing with Milli-Q water. The slides were then stored under nitrogen at 4° C. until use.

Hybridization with KRAS G12D target DNA: The target solutions (Integrated DNA Technologies Inc., USA) were prepared by serial dilution using 2×SSPE buffer (pH 7.4) (Sigma-Aldrich) containing 0.2% SDS. A sample solution of 450 zM (450×10⁻²¹ M) was prepared. The sample solution was heated to 95° C. for 3 min, and 40 μL of the solution was incubated on a capture probe spotted glass slide at 50° C. for 24 h using a microarray hybridization kit (Agilent Technologies) and a hybridization oven. After hybridization, the slide was washed with 0.2× SSPE buffer containing 0.02% SDS (pH 7.4) at 60° C. for 20 min. Finally, the slide was rinsed with 0.2× SSC buffer (pH 7.4) at room temperature followed by washing with PBS buffer (pH 7.4).

For control experiments and to assess the specificity of the current approach, solutions of WT KRAS DNA (Integrated DNA Technologies Inc., USA) were prepared by serial dilution with 2× SSPE buffer containing 0.2% SDS (pH 7.4). Additionally, a solution containing WT DNA at 45 aM was prepared. A solution containing the respective amount of WT KRAS DNA along with 450 zM of KRAS G12D DNA was subjected to hybridization following the above protocol.

Protein extraction and purification: Cloned E. coli MutS with an N-terminal His-tag in a pET15b vector was overexpressed from the E. coli strain BL21 (DE3) (Novagene). The protein was sequentially purified by a Hi-trap Ni-column (Amersham Pharmacia Biotech) and a MonoQ column (Amersham Pharmacia Biotech).

Preparation of MutS-tethering AFM tip: The dendron (27-acid dendron)-coated AFM tips (Si₃N₄, DPN pen-type B, NanoInk Inc., USA) were obtained from NB POSTECH, Inc. The AFM tips were placed into an acetonitrile solution of bis(NHS)PEG₅ (Thermo Scientific, USA) (25 mM) and N,N-diisopropylethylamine (DIPEA) (1.0 mM) for 3 h at room temperature. After the reaction, the tips were placed in a stirred DMF solution for 30 min to remove nonspecifically bound molecules. Next, the tips were washed with methanol and dried under vacuum for 30 min. The NETS-activated AFM tips were then treated with 10 mM nitrilotriacetic acid (NTA) solution in 5 mM sodium bicarbonate solution for 15 h at room temperature. Subsequently, the tips were rinsed with 5 mM sodium bicarbonate solution to remove the excess unreacted molecules and were then placed into a 50 mM solution of nickel chloride for 4 h at room temperature. The tips were rinsed with a brine solution and allowed to react in a 200 nM solution of histidine-tagged MutS in PBS (pH 7.4) buffer for 2 h at room temperature. Finally, the tips were washed with PBS followed by Milli-Q water and were stored under PBS at 4° C. until use.

Hybridization of HD780 cfDNA reference standard set: A standard reference cfDNA set (HD780) was used (Horizon Discovery, UK). The cfDNA samples were derived from human cell lines and were fragmented to an average size of 160 bp. The standard set contained single-nucleotide variants (SNPs/SNVs) of eight mutations. The sample set comprised four vials with allele frequencies of 5%, 1.0%, 0.1% and 100% wild type: vials with 1.0%, 0.1% and 100% wild type allele frequencies were used. For the 1.0% allele frequency, 0.3 μL of the sample solution was mixed with 3.6 μL of LNA/DNA blocker (c=10 aM), and the mixture was diluted to 40 μL. For the 0.1% allele frequency, both 1.0 μL and 0.6 μL of the sample solutions were mixed with 3.6 μL of LNA/DNA blocker (c=10 aM), and the mixture was diluted to 40 μL. In the case of 100% wild type allele frequency, 1.0 μL of the sample solution and the above amount of the LNA/DNA blocker was diluted to 40 μL. For all cases, the sample solution was heated to 95° C. for 3 min and then placed onto the capture probe spot as per the protocol described previously.

Extraction of cfDNA from plasma: Peripheral blood samples were drawn in ethylenediaminetetraacetic acid (EDTA)-containing tubes from 14 patients who were diagnosed and treated pancreatic cancer in Seoul St. Mary's Hospital. Plasma was separated within one hour of collection through two centrifugation steps: 2,000×g at 4° C. for 10 minutes, followed by 16,000×g at 4° C. for 10 minutes. cfDNA were extracted using the QIAamp Circulating Nucleic Acid kit (Qiagen, Hilden, Germany) with QIAvac 24 Plus system (Qiagen) according to the manufacturer's instructions. Then, DNA concentration was measured by the fluoroscopic quantification using Qubit 3.0 Fluorometer (Thermo Fisher Scientific, Waltham, MA USA) with Qubit DsDNA HS Assay Kit (Qiagen). The study was conducted in accordance with the Declaration of Helsinki, and was approved by the Institutional Review Board/Ethics Committee of Seoul St. Mary's Hospital (IRB No. KC18TESI0701).

BEAMing and ddPCR: Sysmex Inostics BEAMing Digital PCR (Sysmex Inostics GmbH, Hamburg, Germany) was used with OncoBEAM™ RAS CRC kit RUO (ZR150001) and CyFlow Cube 6i and Robby instruments according to manufacturer instructions. Pre-amplification was performed with 123 μL of isolated cfDNA. Data were analyzed using the BEAMing software (Sysmex Inostics GmbH).

ddPCR™ Mutation Detection Assays, Validated (#10049550, Bio-Rad, Hercules, CA) was used for the KRAS G12D analysis according to manufacturer's instructions. 8 μL of cfDNA diluent was mixed with 2 μL of ddPCR Mut Assay KRAS G12D and 10 μL of Supermix for Probes (Bio-Rad, #31863024). Droplets were generated with QX200 ddPCR system and analyzed with QuantaSoft (Bio-Rad, version 1.7.4.0917). All experiments were duplicated, separately.

Quantitative imaging with AFM and data analysis: All force mapping experiments were performed with a NanoWizard 3 AFM (JPK Instrument, Germany) in quantitative imaging (QI) mode. The spring constant of each cantilever was calibrated via the thermal fluctuation method, and the spring constant values were within the range of 0.01 N/m to 0.03 N/m. A tip velocity of 18 μm/sec was employed during scanning with a z-length of 200 nm. The tip was programmed to approach the surface with a contact force of 80 pN to minimize sample damage. To visualize the individual surface-captured target molecules, high-resolution QI maps were recorded within area of 150×150 nm² or 200×200 nm². Scanning of an entire 2 μm diameter spot of was conducted with 200×200 pixels. To ensure scanning of the entire spot area, the scanning area was adjusted for larger spots. For the latter case, a higher pixel number was adopted to keep the pixel size (10×10 nm²) constant. All AFM measurements were performed in PBS buffer (pH 7.4) at room temperature.

A total of 40,000 F-D curves recorded for each QI map (200×200 pixels) were analyzed with the JPK data processing program. First, the recorded F-D curves were filtered to select only those with appropriate adhesion forces (≥18 pN, ≤40 pN) and stretching distances (5-35 nm). Next, a linear fitting script was implemented in Jython to identify the specific force curves with appropriate nonlinear stretching prior to the unbinding event. Individual specific adhesion force maps were then generated, and three successive maps were overlaid after drift correction using an in-house MATLAB program. Median filter was applied to the overlaid adhesion maps to distinctly identify the positive clusters by reducing the scattered pixels. In addition, a MATLAB script was used to calculate the cluster radius, identify the qualified clusters from the obtained overlaid QI maps, and calculate the cluster number.

Results

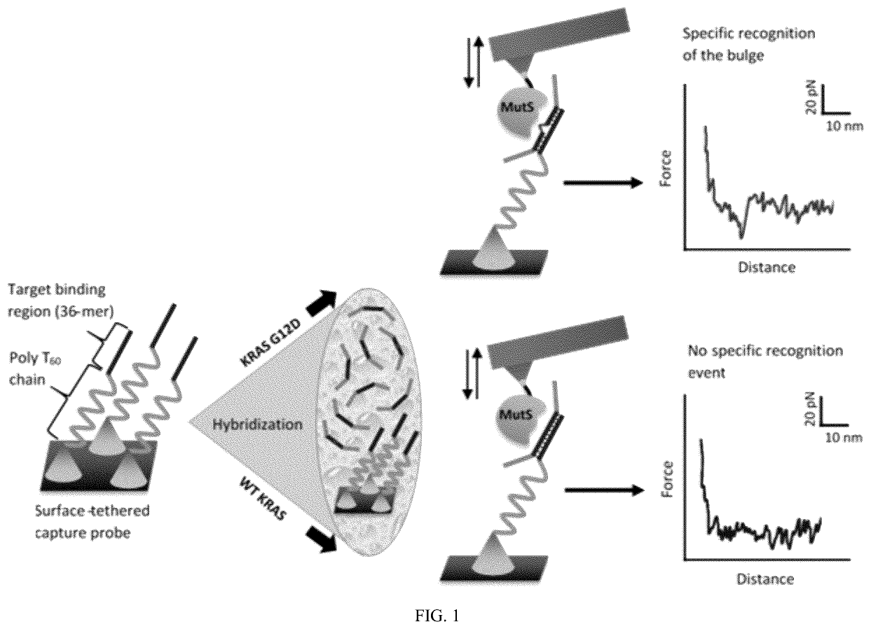

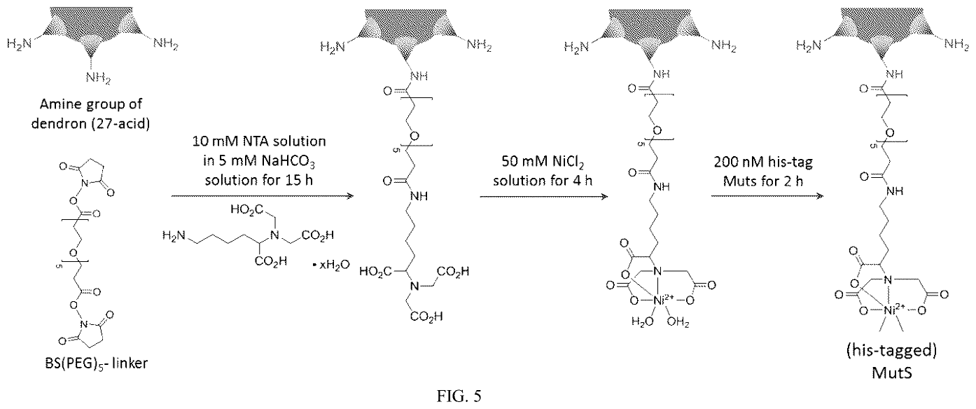

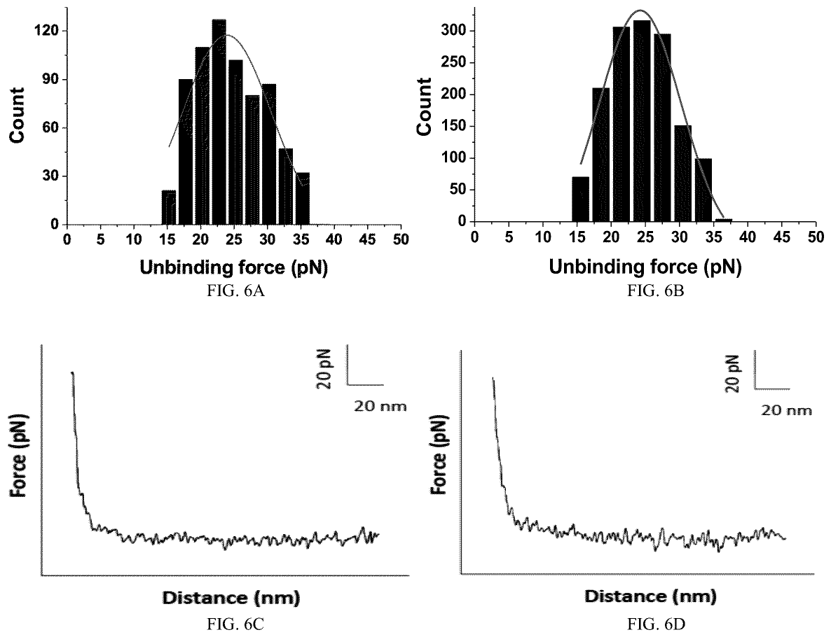

MutS-tethering AFM tip specifically recognizes the captured mutated KRAS DNA: Experiment was designed to sense KRAS-mutated DNA using MutS-tethered AFM tips (FIG. 1). MutS is a DNA mismatch repair protein that recognizes and binds to heteroduplex DNAs containing mispaired or unpaired bases (insertions/deletions) of 1-4 nucleotides. MutS is stable from pH 1.5 to 12 at 25° C. and at neutral pH up to 80° C. MutS has variable affinities for different mismatches and forms the strongest complexes with GT mismatches and single unpaired bases. For tethering, dendron (27-acid dendron)-coated AFM tips were treated with bis(NHS)PEG₅ solution in acetonitrile to generate an NHS group at the apex of the dendrons. The activated AFM tips were then treated with a chelator, nitriloacetic acid (NTA), followed by Ni(II) ion complexation to form NTA-Ni(II). Next, histidine-tagged MutS was immobilized through the binding of histidine with the complexed Ni(II) ion (FIG. 5). To capture KRAS-mutated DNA in solution on a spot on the surface, an amine-terminated capture probe that was fully complementary to the wild-type (WT) KRAS sequence was immobilized onto a glass surface. Therefore, KRAS-mutated DNA (KRAS G12D) and WT DNA hybridized with the capture probes: the former generated a bulged duplex, whereas the latter formed a perfectly matched DNA duplex. Both 156-mer custom-made DNAs with identical sequences to WT KRAS and KRAS G12D mutation, respectively, were used as the targets. The length of the capture probe (36 nt) was chosen such that the melting temperature difference between the WT target and the mutated target remained minimal upon hybridization. Therefore, the competitive preference of the WT target over the mutated target during hybridization can be minimized at lower hybridization temperature. In this respect, the hybridization temperature (50° C.) was approximately 30° C. lower than the respective melting temperatures (81.4° C. and 80.3° C.), which ensured a satisfactory hybridization rate. The specific adhesion event between MutS protein and on-surface mismatched DNA duplex was observed reproducibly with AFM. By contrast, the fully matched duplex, arising from the hybridization of WT DNA with the capture probe, remained silent during the force measurement. The specific nature of MutS protein recognizing the bulge of the DNA duplex was confirmed at the single-molecule level, which allowed visualization of only the mutated DNAs captured on the surface, even in the presence of an excess amount of captured WT DNA. In addition to the single point mutation, MutS-tethered AFM tip could detect mismatched/deleted duplexes up to four bases. The most probable adhesion force values for the case of triple-base mismatches/deletions are similar to that for the case of single-base mismatch (FIGS. 6A and 6B), whereas such a specific event was not observed for the case of five-base mismatches or five-base deletions (FIGS. 6C and 6D, respectively). Therefore, the interaction between MutS and the corresponding DNA duplexes at the single-molecule level matches the ensemble-averaged observation.

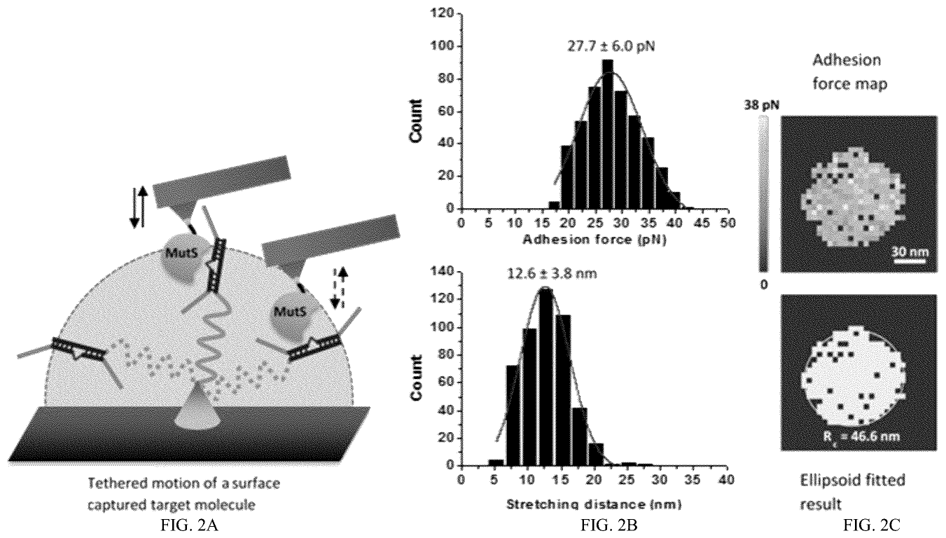

Miniaturized capture probe spots were fabricated via FluidFM technology. See, for example, Gruter, R. R.; Voros, J.; Zambelli, T. Nanoscale 2013, 5, 1097-1104. A microchanneled cantilever equipped with a pyramidal tip with a 300 nm aperture was used to spot the capture probe onto a photolithographically etched and activated glass slide at known (x, y) coordinates. Typical spot diameters were within the range of 1.5-2.4 μm to ensure scanning of the entire area at high resolution for the detection of mutant alleles present in very low frequency. The entire probe spot surface was scanned with a MutS-tethered AFM tip in QI mode, and the F-D curves were collected at every pixel. Without being bound by any theory, it is believed that the hydrodynamic radius of the surface-captured target molecules is important because the MutS protein can interact with the on-surface mismatched DNA duplex only within the area characterized by the hydrodynamic radius of the surface-captured molecules, and such information provides a reasonable pixel size that makes each captured DNA a cluster of pixels in a map (FIG. 2). Therefore, a understanding of the hydrodynamic radius of the surface-captured target molecules is desired to determine the optimal pixel size for scanning. Whereas an excessively large pixel size tends to miss the target, the time to examine the entire area increases when the pixel size is too small. Furthermore, it is difficult to avoid false pixels in which the F-D curves are very similar to those of the specific event. By the nature of randomness, such pixels are scattered within the scan area and do not form clusters.

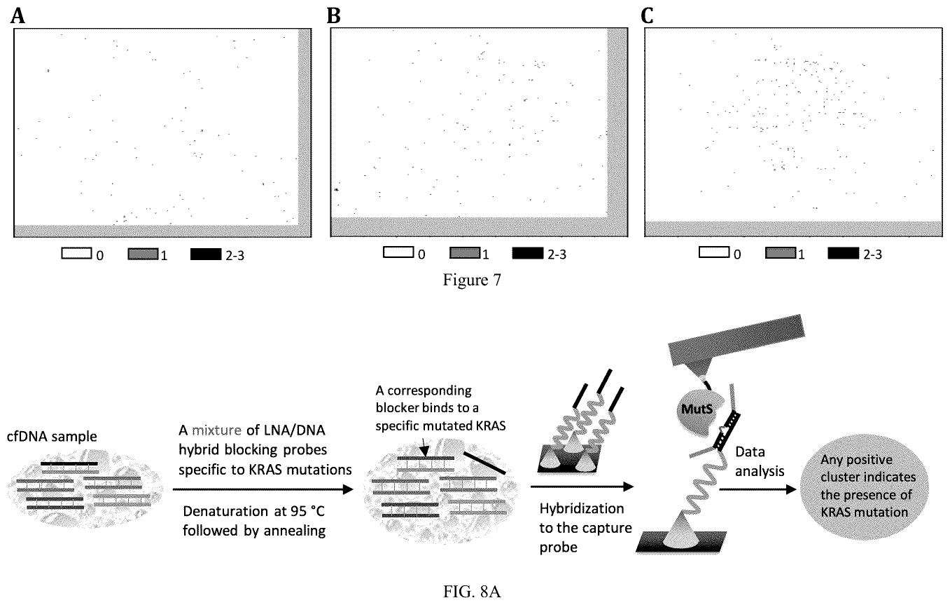

A pixel size of approximately ½ of the hydrodynamic radius gave a cluster of approximately ten positive pixels, and this resolution is sufficient to unambiguously locate individual true targeting DNAs. At such resolution, the cluster size is one of the key factors in judging the qualification. A 156-mer custom-made KRAS G12D mutated DNA was used as the target probe, which formed a singly mismatched DNA duplex upon hybridization with the capture probe (Table 1). When the MutS-tethering AFM tip approached the surface, the MutS protein formed a noncovalent complex with the mismatched duplex, and upon retraction of the tip, unbinding of the complex occurred. To determine the hydrodynamic radius, high-resolution adhesion force maps were collected via QI mode every 5 nm. The specific F-D curves with a nonlinear stretching profile prior to the unbinding were collected for statistical analysis, and the most probable adhesion force and stretching distance were obtained. The values of the most probable adhesion force and stretching distance were obtained from three different locations, and the average values were 26.2±4.4 pN and 14.4±5.3 nm, respectively (see FIG. 2B for a case). The adhesion force was well within the range reported for protein-ligand pairs and was sufficiently large to be distinguishable from background noise. The observed adhesion force can be attributed to the unbinding of the complex between the MutS protein and surface-captured mismatched DNA duplex because the unbinding force between His₆ and Ni(II) is 525±41 pN. The circular shape and size of the clusters reflect the motion of the tethered DNA in two-dimensional space. The low frequency of the specific events and the absence of such qualified clusters for the fully matched surface-captured DNA duplex, LNA-DNA duplex, and ssDNA capture probe further confirmed the specificity of MutS protein (FIG. 7).

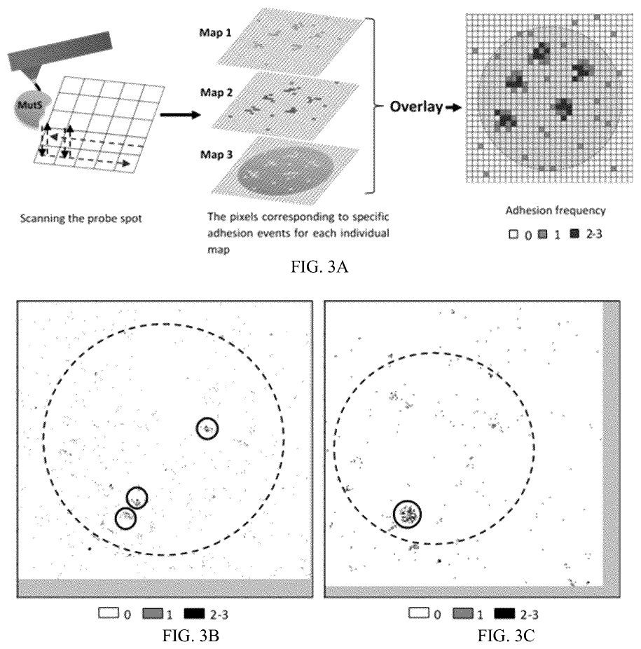

Finding qualified positive clusters: First, the hydrodynamic radius of the surface-captured target molecules was estimated via ellipse fitting. Three different areas were scanned at high resolution (5 nm pixel size, QI mode) and collected three to six maps at each location. Three consecutive maps were then superimposed after drift compensation to generate one or two overlaid maps for each location (FIG. 3A). The average cluster radius was estimated to be 40.3 nm, in agreement with the geometric configuration of the surface-captured target molecule.

With the given cluster radius, the optimal pixel size was determined to scan the entire probe spot area to visualize the individual surface-captured target molecule. Scanning a 2 μm diameter spot every 10 nm requires 16 min to complete one map. Typically, a slightly larger area than the spot size was examined to ensure scanning of the entire spot. Therefore, it took 48 min in total to generate three successive maps to visualize all and individual captured target DNAs on the surface.

The topography, slope and adhesion maps were simultaneously recorded during the QI. The F-D curves were screened to select only those with appropriate adhesion forces (≥18 pN, ≤40 pN) and stretching distances (5-35 nm). 2D images were then generated to illustrate the positive pixels attributed to such specific F-D curves. Three consecutive specific adhesion maps were then overlaid after correcting for the lateral drift with an in-house MATLAB program. In the overlaid specific adhesion map, pixels where a single specific adhesion event was detected are colored in green, and pixels with two or three specific adhesion events are colored in red. To check whether this approach works for clinical samples, the qualified clusters for KRAS G12D mutated DNA of 10 copies were counted in the presence of varying amounts of WT DNA. Whereas 3.7 clusters were observed in the absence of WT DNA, 4.3 clusters were observed in the presence of 1,000 copies of WT DNA (Table 2). The cluster number was reduced in the presence of more than 10,000 copies of WT DNA. Nevertheless, it was possible to detect more than one cluster in the presence of 100,000 copies of WT DNA. Moreover, competitive binding of WT DNA to the capture probe is not prevalent in clinical samples because they are in the double-stranded form (Vide infra). Multiple runs of a control experiment conducted with WT DNA found that the occurrence of red pixels was rare and that the largest cluster observed in all cases was too small to be qualified (FIG. 7A). Based on these results, the criteria was defined to assign qualified clusters truly reflecting the surface-captured KRAS-mutated DNA. First, the cluster radius must exceed 30 nm. Second, the qualified cluster must contain at least one pixel where the specific event was observed repeatedly. Although the cut-off cluster radius was smaller than the hydrodynamic radius measured at 5 nm resolution, the above selection criteria was consistently followed throughout this study because maps of 10 nm resolution for detection was used.

TABLE 2
Detection of KRAS G12D in the presence
of varying amounts of WT KRAS genes.

| Ratio of KRAS | Observed number of clusters in a spot | | | | |
|---|---|---|---|---|---|
| G12D DNA to | (R_{c} ≥ 30.0 nm) | | | | |
| WT KRAS DNA | spot 1 | spot 2 | spot 3 | mean | SD |
| 10:0 | 3 | 2 | 6 | 3.66 | 2.08 |
| 10:1000 | 6 | 3 | 4 | 4.33 | 1.50 |

Use of LNA/DNA chimeric blocking probes: As the method outlined is applicable to single-stranded targets in a sample solution, an additional measure may be necessary to make this approach viable for clinical samples. Heating the DNA duplexes (95° C.) in the presence of the surface-immobilized capture probe did not lead to any detectible hybridization, presumably because the resulting single-stranded target rebound to its complementary counterpart before it bound to the capture probe. Therefore, a blocker (or a blocking probe) was used to inhibit rebinding. The blocking probe should be highly specific and effective to capture a mutated gene present with a very low mutant allele frequency. Moreover, the blocking probe should favor binding with the complementary strand during the annealing step so that the binding leaves the target DNA free. Additionally, the blocking probe must bind preferentially to the above strand that is complementary to the mutated gene rather than the WT DNA. Such a preference allows the use of a small amount of blocking probe. Locked nucleic acid (LNA) was selected to achieve these goals, as the thermal stability of a duplex can be enhanced by +2° C. to +8° C. per substitution of LNA monomer, depending on the length and sequence of the probe.

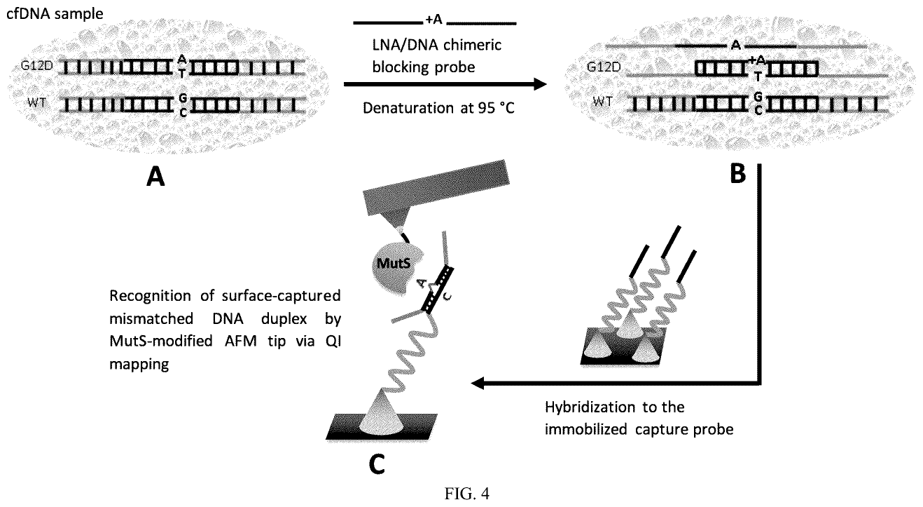

A 36-mer LNA/DNA chimeric blocking probe was designed to increase the melting temperature difference between the newly formed duplex (FIG. 4, step B) and the native target duplex (KRAS G12D, FIG. 4, step A) (Table 1). About 40% of the bases of an oligonucleotide was substituted with LNAs, and runs of more than four consecutive LNA bases was avoided. The melting temperature of the newly formed duplex with a 36-mer LNA/DNA chimeric blocking probe was estimated to be 95.2° C. (melting temperature prediction tool, Exiqon), whereas that of the pristine duplex of the 156-mer KRAS G12D gene was estimated to be 85.5° C. (IDT olio analyzer tool, 298 mM [Na⁺]. About 900 zM (40 μL, ˜20 copies) of the blocking probe was added to the sample solution prior to the denaturation step (95° C. for 3 min). During annealing, the formation of a stable duplex between the LNA/DNA chimeric blocking probe and DNA complementary to the target resulted in the single-stranded mutated target being free to bind to the capture probe, and the resulting duplex was eventually recognized by MutS (FIG. 4, step C). Formation of the singly mismatched duplex was ensured via hybridization for 24 h at 50° C., which is 30° C. below the melting temperature of the respective duplex. The binding of the LNA/DNA chimeric blocking probe to the single-stranded DNA originating from the denaturation of WT dsDNAs was less likely because it would result in a single mismatch and LNA boasts high discriminating ability with respect to SNPs. For a 12-mer LNA probe, the melting temperature difference (ΔTₘ) between a fully matched and a singly mismatched duplex is 21.5° C. Because of the outnumbered WT DNA in clinical samples, there is a possibility of generating some free single-stranded WT DNA, but the duplex formed with the capture probe on the surface is not recognized by MutS protein. In addition, the unreacted blocking probe can hybridize with the capture probe, but MutS cannot recognize the mismatched duplex due to the involvement of the synthetic LNA moieties.

Detection of KRAS G12D mutated DNA in a cfDNA reference standard set: The validity of the approach was tested with Horizon's HD780 cfDNA reference standard set. The cfDNA products were derived from human cell lines and fragmented to an average size of 160 bp, which is close to that of cfDNA extracted from human plasma. The set includes multiple engineered single-nucleotide variants (SNVs/SNPs) with eight mutations at 5.0%, 1.0% and 0.1% allele frequencies. Samples were examined with 1.0% and 0.1% allele frequencies to estimate the sensitivity of the current approach, and samples with 100% WT cfDNA were investigated to examine the specificity (Table 3). For the sample with a 1.0% allele frequency, 0.30 μL of the sample solution (ca. 10 copies) was mixed with the LNA/DNA chimeric blocker (20 copies) and diluted to a final volume of 40 μL. The solution was then denatured at 95° C. for 3 min, followed by hybridization to the capture probe at 50° C. for 24 h. Identical experimental steps were followed by diluting 1.0 μL and 0.6 μL of the sample solutions, respectively, of 0.1% allele frequency (ca. 5 copies and ca. 3 copies) and 1.0 μL of the sample solution of 100% WT cf DNA to 40 μL in the presence of the blocker (20 copies) in each case.

TABLE 3
The number of clusters observed from each capture probe
spot for various allele frequencies. For all cases,
900 zM of LNA/DNA chimeric blocking probe was added
to the sample solution prior to denaturation.

| | Observed No. of Clusters in a Spot | | | | |
|---|---|---|---|---|---|
| | (R_{c} ≥ 30.0 nm) | | | | |
| CfDNA Ref. Std. Sample | Spot 1 | Spot 2 | Spot 3 | Mean | SD |
| 1.0% multiplex (~10 copies) | 7 | 5 | 5 | 5.66 | 1.15 |
| 0.1% multiplex (~5 copies) | 3 | 1 | 2 | 2.00 | 1.00 |
| 0.1% multiplex (~3 copies) | 1 | 1 | 1 | 1.00 | 0.00 |
| 100% multiplex (WT cfDNA | 0 | 0 | 0 | 0.00 | 0.00 |
| 0 copies) | | | | | |

* Experiments were repeated 10 times, and the same results were obtained for each run.

For the samples with 1.0% and 0.1% allele frequencies, three replicates were performed for each case, and for the samples with 100% WT multiplex, the experiments were repeated ten times (Table 3). For the samples with 1.0% and 0.1% allele frequencies, qualified clusters were always observed in all replicate experiments (FIGS. 3B, C and Table 3), and the mean cluster counts were 5.66 and 2.00, respectively. Furthermore, experiments with the reduced sample volume (0.6 μL) of 0.1% allelic frequency resulted in the detection of the qualified cluster (FIG. 3C) (mean cluster count=1.0). The presence of qualified clusters for all cases with 1.0% and 0.1% allele frequencies indicates high sensitivity. Additionally, the absence of qualified clusters for all ten runs with 100% WT cfDNA confirmed the high specificity of the approach.

Detection of EGFR L858R mutated DNA in a cfDNA reference standard set: To assess the applicability for other mutation types, detecting EGFR L858R mutated DNA in cfDNA samples was examined, as it is one of the most commonly accounted EGFR mutations in the non-small cell lung cancer (NSCLC). The cfDNA reference standard set, a corresponding capture probe, and a relevant blocker were employed (Table 1). For all the samples, after adding the LNA/DNA chimeric blocker (20 copies), the sample solution (1.0 μL) was diluted to a final volume of 40 μL. Next, the sample solution was denatured at 95° C. for 3 min, and the hybridization to the surface immobilized capture probe was followed at 50° C. for 24 h. For each sample, the experiments were repeated three times (Table 4). For samples with 5.0%, 1.0% and 0.1% allele frequencies, the qualified clusters were always detected for all the runs, and the mean number of the clusters were 13.33, 10.66 and 1.66, respectively. It is worth mentioning that for the samples with 5.0% and 1.0% allele frequencies, the observed number of the cluster were saturated, as we added the fixed amount of the blocker (20 copies) for all the experiments. For WT cfDNA samples, no cluster was observed for all the replicate runs, which validates the high specificity of the approach. In addition, the results obtained with the sample of 0.1% allele frequency reaffirmed the characteristics. In the reference standard set, whereas four copies of EGFR mutant variant were present in 1.0 μL, numbers of the coexisting variants (L858R, T790M, delE746-A750, and V769-D770insASV) were 4, 3, 2, and 2, respectively. With the above capture probe and blocker designed for L858R mutation, we observed that the qualified cluster count was 1.7 in average. The number close to two demonstrates that the approach is highly specific and capable of discriminating the other mutant variants effectively.

TABLE 4
The number of clusters observed from each capture probe spot for
the detection of EGFR L858R mutant in cfDNA samples of various
allele frequencies. For all the cases, 900 zM (20 copies) of
LNA/DNA chimeric blocker was added prior to denaturation.

| | Observed number of clusters in a spot | | | | |
|---|---|---|---|---|---|
| cfDNA | (R_{c} ≥ 30.0 nm) | | | | |
| reference standard sample | spot 1 | spot 2 | spot 3 | mean | SD |
| 0.1% multiplex (1.0 μL of the | 2 | 1 | 2 | 1.66 | 0.57 |
| sample was diluted to 40 μL) | | | | | |
| (4 copies) | | | | | |
| 1.0% multiplex (1.0 μL of the | 12 | 9 | 11 | 10.66 | 1.52 |
| sample was diluted to 40 μL) | | | | | |
| (40 copies) | | | | | |
| 5.0% multiplex (1.0 μL of the | 17 | 13 | 10 | 13.33 | 3.51 |
| sample was diluted to 40 μL) | | | | | |
| (203 copies) | | | | | |
| 100% multiplex (WT cfDNA) | 0 | 0 | 0 | 0.00 | 0.00 |
| (1.0 μL of the sample was | | | | | |
| diluted to 40 μL) (0 copy) | | | | | |

Detection of KRAS mutation at codon 12 in clinical cfDNA samples: The AFM-based approach was applied for cfDNA from peripheral blood plasma of patients with pancreatic cancer. BEAMing was performed to detect and estimate the amount of RAS mutations present in each sample. By employing the former approach, four samples were examined having 0.006%-6.708% of KRAS codon 12 mutations and ten WT samples. The samples were also tested with ddPCR investigating KRAS G12D mutation, and the results were summarized in Table 5.

TABLE 5
Results obtained with the clinical samples from each capture probe spot
for various allele frequencies. BEAMing analyzed KRAS codon 12, and
ddPCR analyzed KRAS G12D. For AFM, 900 zM of LNA/DNA chimeric blocker
(G12D) was added to the sample solution prior to denaturation. The
cluster numbers were obtained from independent duplicated runs.

| | Method | CMC01 | CMC02 | CMC03 | CMC04 |
|---|---|---|---|---|---|
| BEAMing | Mutant fraction of codon 12 | 6.708% | 0.230% | 0.126% | 0.006% |
| | Number of mutant beads | 24,535 | 710 | 601 | 14 |
| ddPCR | Mutant fraction of G12D | 17.72% | 0.64% | 0.33% | 0.00% |
| AFM | Sampled volume/copy number | 0.10 μL/20 | 1.0 μL/5.7 | 1.0 μL/4.8 | 5.0 μL/0.6 |
| | Number of clusters of G12D | 11.0 | 3.5 | 3.0 | 0.0 (2.0)¹⁾ |

¹⁾By use of CMC01 sample and the reference standard set, a sample of the same allele frequency as CMC04 was prepared, and two clusters were observed for 40 μL (5 copies) consistently out of two runs.

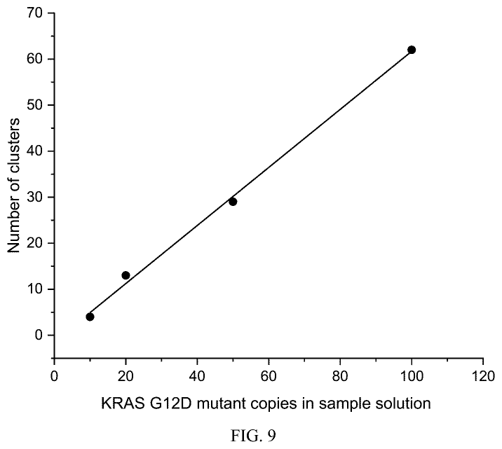

For G12D, 11.0, 3.5, and 3.0 clusters were observed, respectively, when 0.1, 1.0, and 1.0 μL of samples CMC01, CMCO2, and CMC03 were taken so that the solutions contain 20, 5.7, and 4.8 copies. Whereas zero cluster was observed when 5.0 μL of sample CMC04 (the copy number=0.6) was examined, it is reasonable because the copy number is far below single. For comparison, 0.015 μL of sample CMC01 (5 copies) was mixed with WT cfDNA standard sample to compare with the LOD of BEAMing. Two replicate runs showed two positive clusters for each. The result demonstrates that the LOD of the present invention is comparable to BEAMing. In addition, a high degree of correlation (R²=0.995, linear regression model) was observed between the numbers of mutant copies (KRAS G12D) present in the sample solution and the detected clusters (FIG. 9). The samples containing up to 100 mutant copies were examined in the presence of a fixed amount of LNA/DNA blocker (200 copies) in each case.

It is noteworthy that the methods of the present invention do not require to set the cut-off value that leads to distorted copy number (or allele frequency) in PCR based methods such as ddPCR. For ten negative clinical samples examined with the former approach showed that number of the cluster always turned out to be zero. This example clearly demonstrated high specificity and high selectivity of methods of the present invention.

### DISCUSSION

An amplification-free direct method to detect a gene mutation at very low allele frequencies in cfDNA samples via force-based AFM was demonstrated using a DNA mismatch repair protein (e.g., MutS). In some embodiments, an LNA/DNA chimeric blocking probe was employed to ensure that the target DNA (e.g., a single stranded mutated gene) was free for the capture probe on the surface. The inherent specificity of a DNA mismatch repair protein was exploited at the single-molecule level. In addition to the characteristic melting behavior of the LNA/DNA chimeric blocking probe, the choice of unnatural blocking is advantageous because the duplex formed with the blocking probe is silent with respect to DNA mismatch repair proteins, such as MutS.

Some of the key features and advantages of the present invention are the sensitivity/specificity and corresponding limit detection (LOD). In some embodiments, a sensitivity/specificity close to 100% (e.g., 0/28 false negatives, 0/23 false positives) was achieved. Such enhancement may be attributed to the nature of the measurement (a direct quantification without amplification, labeling, or modification) and highly specific DNA mismatch repair protein, such as MutS. Repeated detection of the mutated DNA for the sample of 0.6 μL with 0.1% allele frequency (˜3 copies) with 0.015 μL of sample CMC01 and with 5.0 μL of sample CMC03 indicates that the LOD at least as comparable to that of the current most sensitive methods. For a single test with the current AFM approach, a blood of 1 mL must be sufficient for the sample down to 0.1% allele frequency. For example, the successful test with the diluted sample CMC01 to make 0.006% allele frequency indicates that a blood of 0.5 mL is large enough with the current AFM approach. In addition to the KRAS G12D mutation, methods of the invention can be used to detect other common KRAS-mutated DNAs related to cancers in cfDNA samples via the use of suitable blocker(s) (FIG. 8A). The unknown KRAS mutation type in cfDNA samples can also be elucidated via this approach by employing a suitable blocker for each type of mutation (FIG. 8B). At the same time, methods and apparatuses of the invention can be used to detect any types of the mutation (codons 12 and 13) if a mixture of the blockers corresponding to G12D, G12V, G12C, G12A, G12S, G12R, and G13D are used. Apart from KRAS mutation, methods and apparatuses of the invention has also been validated for the detection of other mutations (e.g., EGFR mutation) in cfDNA samples. Accordingly, methods and apparatuses of the invention can be used for the detection of point mutation of various other gene and their mutation types. Methods and apparatuses of the invention provide new avenues for analyzing circulating tumor DNA. Superb LOD, sensitivity and specificity close to 100% are some of the key features of the present invention.

Conclusion. Methods and apparatuses of the invention provide direct approach (i.e., without the need for labeling, amplifying, or modifying the sample) to detect a gene mutation (e.g., KRAS-mutated DNA) present with low allele frequency. The use of force-based AFM and a DNA mismatch repair protein (e.g., MutS) tethered AFM tips achieved superb LOD and sensitivity/specificity. These characteristics can be attributed to the nature of AFM, the inherent nature of DNA mismatch repair protein (such as MutS), and the absence of amplification, labeling, or modification steps. The mutated DNA was detected in the clinical cfDNA samples with 6.7-0.006% mutant allele frequency, and a linear response was observed up to 100 copies. The use of the LNA/DNA chimeric blocker has been shown to be effective in ensuring that the target DNA is free to bind to the capture probe on the surface.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A method for directly diagnosing a gene mutation in a subject, said method comprising:
contacting a sample obtained from a subject with a solid substrate comprising a probe oligonucleotide under conditions sufficient to form a target-probe oligonucleotide duplex when a target oligonucleotide is present in said sample, wherein said probe oligonucleotide comprises a complementary oligonucleotide sequence of a wild-type gene;
measuring a level of interaction between said target-probe oligonucleotide duplex and a DNA mismatch repair protein using an atomic force microscope (AFM); and
analyzing said level of interaction to determine the presence of a mismatched target-probe oligonucleotide duplex,
wherein the presence of mismatched target-probe oligonucleotide duplex is indication of a presence of a gene mutation in said subject.

2. The method according to claim 1, wherein said probe oligonucleotide comprises a complementary oligonucleotide of a wild-type gene selected from the group consisting of Ras, BRAF, EGFR, RET, and PIK3CA.

3. The method according to claim 1, wherein said Ras gene is selected from the group consisting of KRas, HRas, NRas, R-Ras, M-Ras, E-ras, Di-Ras1, Di-Ras2, NKIRas1, NKIRas2, TC21, Rap1, Rap2, Rit1, Rit2, Rem1, Rem2, Rad, Gem, Rheb1, Rheb2, Noey2, R-Ras, Rerg, RalA, RalB, RasD1, RasD2, RRP22, RasL10B, RasL11A, RasL11B, Ris/RasL12, and FLJ22655.

4. The method according to claim 2, wherein said method detects a mutation in codon 12 or 13 of KRas gene.

5. The method according to claim 2, wherein said method detects BRAF V600E mutation or RET M918T mutation.

6. The method according to claim 1, wherein said sample comprises cell free DNA (cfDNA).

7. The method according to claim 1, wherein said step of contacting said sample with said solid substrate further comprises the step of contacting said sample with a blocking probe under conditions sufficient to selectively form a blocking probe-mutated gene duplex and a single stranded mutated gene, when said mutated gene is present in said fluid sample.

8. The method according to claim 7, wherein said blocking probe comprises locked-nucleic acid/DNA (“LNA/DNA”) chimeric blocking probe.

9. The method according to claim 8, wherein a melting temperature difference between a LNA/DNA chimeric blocking probe-normal gene duplex and a LNA/DNA chimeric blocking probe-mutated gene duplex is at least 10° C.

10. A method of directly diagnosing for a presence of cancer in a subject, said method comprising:
contacting a fluid sample obtained from subject with a solid substrate comprising a probe oligonucleotide under conditions sufficient to form a target-probe oligonucleotide duplex when a target oligonucleotide is present in said sample, wherein said probe oligonucleotide comprises at least a portion of a wild-type Ras gene; and
analyzing said target-probe oligonucleotide duplex for the presence of a mismatched target-probe oligonucleotide duplex with an atomic force microscope (AFM) comprising a DNA mismatch repair protein attached to a cantilever of said AFM,
wherein the presence of said DNA mismatched target-probe oligonucleotide duplex is an indication that the subject has cancer.

11. The method according to claim 10, wherein said wild-type Ras gene comprises a wild-type KRas gene.

12. The method according to claim 11, wherein said method is used to determine the presence of a mutation in codon 12 or 13 of KRas gene.

13. The method according to claim 12, wherein said method is used to determine the presence of G12D, G12A, G12R, G12C, G12S, G12V, G13D, or a combination thereof.

14. The method according to claim 10, wherein said step of contacting said fluid sample with said solid substrate further comprises the step of contacting said fluid sample with a blocking probe under conditions sufficient to selectively form a blocking probe-mutated Ras gene duplex and a single stranded mutated Ras gene, when said mutated Ras gene is present in said fluid sample.

15. The method according to claim 14, wherein said blocking probe comprises locked-nucleic acid/DNA (“LNA/DNA”) chimeric blocking probe.

16. The method according to claim 15, wherein a melting temperature difference between a LNA/DNA chimeric blocking probe-normal Ras gene duplex and a LNA/DNA chimeric blocking probe-mutated Ras gene duplex is at least 10° C.

17. The method according to claim 10, wherein said fluid sample comprises a cell free DNA (cfDNA).

18. A method for directly screening a subject for cancer, said method comprising:
analyzing a cell free DNA (cfDNA) obtained from subject with a solid substrate comprising a probe oligonucleotide under conditions sufficient to form a target-probe oligonucleotide duplex when a target oligonucleotide is present in said cfDNA, wherein said probe oligonucleotide comprises at least a portion of a wild-type BRAF gene or a wild-type Ras gene; and
analyzing said target-probe oligonucleotide duplex for the presence of a mismatched target-probe oligonucleotide duplex with an atomic force microscope (AFM) comprising a DNA mismatch repair protein attached to a cantilever of said AFM,
wherein the presence of said DNA mismatched target-probe oligonucleotide duplex is used to screen the subject for presence of cancer.

19. The method according to claim 18, wherein said cancer comprises thyroid cancer.

20. The method according to claim 18, wherein said method is used to detect V600E mutation.


SEQUENCE LISTING (6 sequences)

SEQ ID NO: 1 - DNA, 96 bp, synthetic construct
  misc_feature 1..96: note = Synthetic Capture Probe
  misc_feature 1: note = 5'-Cy3
  misc_feature 96: note = 3'-C6-NH2 linker
  source 1..96: mol_type = other DNA
ggcactcttgcctacgccaccagctccaactaccactttttttttttttttttttttttttttttttttttttttttttttttttttttttttttt

SEQ ID NO: 2 - DNA, 156 bp, synthetic construct
  misc_feature 1..156: note = Synthetic 156-mer of WT KRAS gene
  source 1..156: mol_type = other DNA
atagtcacattttcattatttttattataaggcctgctgaaaatgactgaatataaacttgtggtagttggagctggtggcgtaggcaagagtgccttgacgatacagctaattcagaatcattttgtggacgaatatgatccaacaatagaggta

SEQ ID NO: 3 - DNA, 156 bp, synthetic construct
  misc_feature 1..156: note = Synthetic 156-mer of KRAS G12D mutation
  source 1..156: mol_type = other DNA
atagtcacattttcattatttttattataaggcctgctgaaaatgactgaatataaacttgtggtagttggagctgatggcgtaggcaagagtgccttgacgatacagctaattcagaatcattttgtggacgaatatgatccaacaatagaggta

SEQ ID NO: 4 - DNA, 36 bp, synthetic construct
  misc_feature 1..36: note = Synthetic LNA/DNA chimeric blocker
  modified_base 5: mod_base = OTHER; note = Locked nucleic acid
  modified_base 8..9: mod_base = OTHER; note = Locked nucleic acid
  modified_base 11: mod_base = OTHER; note = Locked nucleic acid
  modified_base 14: mod_base = OTHER; note = Locked nucleic acid
  modified_base 16..18: mod_base = OTHER; note = Locked nucleic acid
  modified_base 20: mod_base = OTHER; note = Locked nucleic acid
  modified_base 23: mod_base = OTHER; note = Locked nucleic acid
  modified_base 26..27: mod_base = OTHER; note = Locked nucleic acid
  modified_base 29: mod_base = OTHER; note = Locked nucleic acid
  modified_base 32: mod_base = OTHER; note = Locked nucleic acid
  source 1..36: mol_type = other DNA
gtggtagttggagctgatggcgtaggcaagagtgcc

SEQ ID NO: 5 - DNA, 96 bp, synthetic construct
  misc_feature 1..96: note = Synthetic capture probe
  misc_feature 96: note = linked to 3AmMO label
  source 1..96: mol_type = other DNA
gcacccagcagtttggccagcccaaaatctgtgatctttttttttttttttttttttttttttttttttttttttttttttttttttttttttttt

SEQ ID NO: 6 - DNA, 36 bp, synthetic construct
  modified_base 5: mod_base = OTHER; note = Locked nucleic acid
  modified_base 8..9: mod_base = OTHER; note = Locked nucleic acid
  modified_base 11: mod_base = OTHER; note = Locked nucleic acid
  modified_base 14: mod_base = OTHER; note = Locked nucleic acid
  modified_base 17..19: mod_base = OTHER; note = Locked nucleic acid
  modified_base 21: mod_base = OTHER; note = Locked nucleic acid
  modified_base 23: mod_base = OTHER; note = Locked nucleic acid
  modified_base 26..27: mod_base = OTHER; note = Locked nucleic acid
  modified_base 29: mod_base = OTHER; note = Locked nucleic acid
  modified_base 32: mod_base = OTHER; note = Locked nucleic acid
  source 1..36: mol_type = other DNA
gatcacagattttgggcgggccaaactgctgggtgc